(19) United States
(12) Patent Application Publication
(10) Pub. No.: US 2025/0090556 A1
(43) Pub. Date: Mar. 20, 2025

(54) Polycannabinoid Compositions and Uses Thereof

(71) Applicants: University of Connecticut, Farmington, CT (US); Ommio Health, Inc., Woodbridge, CT (US)

(72) Inventors: Gregory SOTZING, Storrs-Mansfield, CT (US); Konstantine DRAKONAKIS, Woodbridge, CT (US)

(21) Appl. No.: 18/966,250

(22) Filed: Dec. 3, 2024

Related U.S. Application Data
(63) Continuation of application No. PCT/US2023/067817, filed on Jun. 2, 2023.
(60) Provisional application No. 63/348,566, filed on Jun. 3, 2022.

(51) Int. Cl. A61K 31/00; A61K 31/765; A61K 31/785

(52) U.S. Cl. CPC A61K 31/658; A61K 31/765; A61K 31/785

(57) ABSTRACT

Provided herein are polycannabinoid compositions, uses thereof and methods of manufacturing the same. Polycannabinoid compositions described include polymers of a single cannabinoid type or polymers with a variety of cannabinoids. Such compositions are further described as optionally present as a mixture of longer and short polymers having differential functions as a result of size. Compositions are optionally described for combination with carriers. A variety of conditions are described for treatment with such compositions, including cancer, anxiety, and sleep disturbance.

### CROSS REFERENCE

This application is a continuation of PCT/US2023/067817, filed Jun. 2, 2023, which claims benefit of U.S. Provisional Application No. 63/348,566, filed on Jun. 3, 2022, the disclosures of which are incorporated herein by reference in their entirety.

### BACKGROUND

Cannabinoids are compounds derived from plants including the Cannabis sativa plant, or hemp. There are currently over 100 different cannabinoid compounds isolated from plants and a number of other synthetic, or non-naturally occurring cannabinoids have been produced. Cannabinoids are known for their ability to bind to cannabinoid receptors located throughout the body and have shown bioactivity and the potential to treat a variety of medical conditions. Treatments utilizing cannabinoids as therapeutics have numerous limitations including dosing and effective delivery methods. Accordingly, there is a need for formulations of cannabinoids with increased bioavailability and thermal stability.

### BRIEF SUMMARY

Provided herein are compositions wherein the compositions comprise a plurality of polymers comprising a first polymer and a second polymer, wherein the first polymer comprises a plurality of cannabinoid units, wherein the second polymer comprises up to about 20 cannabinoid units, and wherein the second polymer comprises less cannabinoid units than the number of units of the first polymer. Further provided herein are compositions wherein the second polymer is a bioactive polymer. Further provided herein are compositions wherein the second polymer comprises about 2 to 15 cannabinoid units. Further provided herein are compositions wherein the second polymer comprises up to about 5, 10, 15, or 18 cannabinoid units. Further provided herein are compositions wherein the second polymer is a bioactive polymer. Further provided herein are compositions wherein the plurality of polymers comprises a combination of linear and branched polymers. Further provided herein are compositions wherein the plurality of polymers are branched polymers. Further provided herein are compositions wherein the second polymer has a molecular weight of between about 6 kDa to 8.5 kDa. Further provided herein are compositions wherein the first polymer comprises between about 20 to about 80 cannabinoid units. Further provided herein are compositions wherein the first polymer has a molecular weight of about 8 kDa to about 44 kDa. Further provided herein are compositions wherein the first polymer and the second polymer are blended together. Further provided herein are compositions wherein the first polymer comprises serially linked cannabinoid units. Further provided herein are compositions wherein the second polymer further comprises at least one side chain comprising serially linked cannabinoid units. Further provided herein are compositions wherein the polymer comprises branched linkages of the cannabinoid units. Further provided herein are compositions wherein the polymer comprises an ester linker covalently linking two cannabinoid units. Further provided herein are compositions wherein upon degradation of the ester linker a released cannabinoid is present in an amount sufficient for a phenolic antioxidant activity. Further provided herein are compositions wherein the first polymer and the second polymer comprise between 20 and 50 cannabinoid units combined. Further provided herein are compositions wherein the first polymer comprises up to about 50 cannabinoid units. Further provided herein are compositions wherein the first polymer comprises between 20 and 40 cannabinoid units. Further provided herein are compositions wherein the first polymer comprises between 40 and 50 cannabinoid units. Further provided herein are compositions wherein the first polymer does not comprise a free phenol moiety. Further provided herein are compositions wherein the first polymer comprises an endcapping group. Further provided herein are compositions wherein the endcapping group comprises a singular alcohol functionality. Further provided herein are compositions wherein the endcapping group is cannabichromene (CBC) or cannabinol (CBN). Further provided herein are compositions wherein the endcapping group comprises a diol functionality. Further provided herein are compositions wherein the endcapping group is cannabidiol (CBD) or cannabigerol (CBG). Further provided herein are compositions wherein the endcapping group comprises an alcohol and an acid. Further provided herein are compositions wherein the endcapping group comprises cannabinolic acid (CBNA). Further provided herein are compositions wherein the endcapping group comprises at least one alcohol. Further provided herein are compositions wherein the endcapping group comprises two alcohol functionalities and an acid. Further provided herein are compositions wherein the endcapping group is cannabidioloc acid (CBDA) or cannabigerolic acid (CBGA), or a combination thereof. Further provided herein are compositions wherein the endcapping group is a linear or branched alcohol, or a cannabinoid unit having one hydroxyl group, acid group, or ester group before reaction with the polymer. Further provided herein are compositions wherein the composition, prior to degradation, does not provide for phenolic antioxidant activity. Further provided herein are compositions wherein the composition, prior to release of a cannabinoid unit, comprises a tertiary benzylic position for antioxidant function. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise poly (lactic-co-glycolic) acid (PLGA) or polycaprolactone (PCL), or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the cannabinoid units comprise one or more of the cannabinoids listed in TABLE 1. Further provided herein are compositions wherein the composition further comprises a pH adjusting agent. Further provided herein are compositions wherein the pH adjusting agent is a silica particle. Further provided herein are compositions wherein the composition further comprises a carrier polymer. Further provided herein are compositions wherein the carrier polymer comprises a solid dispersion. Further provided herein are compositions wherein the carrier polymer is selected from starch, polylactic acid, polyhydroxybutanoate, or a combination thereof. Further provided herein are compositions wherein the composition further comprises an active pharmaceutical ingredient (API) for co-administration, to a subject. Further provided herein are compositions wherein the API is administered to the subject in the same composition. Further provided herein are compositions wherein the API is administered to the subject in a second composition. Further provided herein are compositions wherein the composition further comprises a second, biodegradable polymer. Further provided herein are compositions wherein the biodegradable polymer is polylactic acid (PLA). Further provided herein are compositions wherein the polymers are blended. Further provided herein are compositions wherein the API is selected from TABLE 2 or is a combination of APIs listed in TABLE 2. Further provided herein are compositions wherein the polymers effect API release from the composition, as compared to a composition not comprising the polymers. Further provided herein are compositions wherein the composition comprising the polymer further comprises a bioactive polycannabinoid polymer, and wherein the bioactive polycannabinoid polymer has an average molecular weight of less than 50,000 daltons. Further provided herein are compositions further comprising a hydrogel. Further provided herein are compositions further comprising a lipid nanoparticle. Further provided herein are compositions wherein the composition is in a liquid, semiliquid, or solid dosage form. Further provided herein are compositions wherein the composition is in the dosage form of a table, pill, capsule, lotion, lozenge, slurry, or cream.

Provided herein are compositions, wherein the composition comprises: a polymer, wherein the polymer comprises a plurality of cannabinoid units, wherein the polymer comprises at least two chains comprising cannabinoid units, and wherein the polymer is a branched polymer. Further provided herein are compositions wherein the polymer is a bioactive polymer. Further provided herein are compositions wherein the second polymer has a molecular weight of between about 6 kDa to 8.5 kDa. Further provided herein are compositions wherein the first polymer comprises between about 20 to about 80 cannabinoid units. Further provided herein are compositions wherein the polymer comprises an ester linker covalently linking two cannabinoid units. Further provided herein are compositions wherein upon degradation of the ester linker a released cannabinoid is present in an amount sufficient for a phenolic antioxidant activity. Further provided herein are compositions wherein the polymer comprises between 2 to 20 cannabinoid units. Further provided herein are compositions wherein the polymer does not comprise a free phenol moiety. Further provided herein are compositions wherein the polymer comprises an endcapping group. Further provided herein are compositions wherein the endcapping group comprises a singular alcohol functionality. Further provided herein are compositions wherein the endcapping group is CBC or CBN. Further provided herein are compositions wherein the endcapping group comprises a diol functionality. Further provided herein are compositions wherein the endcapping group is CBD or CBG. Further provided herein are compositions wherein the endcapping group comprises an alcohol and an acid. Further provided herein are compositions wherein the endcapping group comprises CBNA. Further provided herein are compositions wherein the endcapping group comprises at least one alcohol. Further provided herein are compositions wherein the endcapping group comprises two alcohol functionalities and an acid. Further provided herein are compositions wherein the endcapping group is CBDA or CBGA, or a combination thereof. Further provided herein are compositions wherein the endcapping group is a linear or branched alcohol, or a cannabinoid unit having one hydroxyl group, acid group, or ester group before reaction with the polymer. Further provided herein are compositions wherein the composition, prior to degradation, does not provide for phenolic antioxidant activity. Further provided herein are compositions wherein the composition, prior to release of a cannabinoid unit, comprises a tertiary benzylic position for antioxidant function. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise PLGA or PCL, or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the cannabinoid units comprise one or more of the cannabinoids listed in TABLE 1. Further provided herein are compositions wherein the composition further comprises a pH adjusting agent. Further provided herein are compositions wherein the pH adjusting agent is a silica particle. Further provided herein are compositions wherein the composition further comprises a second polymer. Further provided herein are compositions wherein the second polymer is a solid dispersion. Further provided herein are compositions wherein the second polymer is a selected from starch, polylactic acid, polyhydroxybutanoate, or a combination thereof. Further provided herein are compositions wherein the composition further comprises an active pharmaceutical ingredient (API) for co-administration, to a subject. Further provided herein are compositions wherein the API is administered to the subject in the same composition. Further provided herein are compositions wherein the API is administered to the subject in a second composition. Further provided herein are compositions wherein the composition further comprises a second, biodegradable polymer. Further provided herein are compositions wherein the biodegradable polymer is polylactic acid (PLA). Further provided herein are compositions wherein the polymers are blended. Further provided herein are compositions wherein the API is selected from TABLE 2 or is a combination of APIs listed in TABLE 2. Further provided herein are compositions wherein the polymers effect API release from the composition, as compared to a composition not comprising the polymers. Further provided herein are compositions wherein the composition comprising the polymer further comprises a bioactive polycannabinoid polymer, and wherein the bioactive polycannabinoid polymer has an average molecular weight of less than 50,000 daltons. Further provided herein are compositions further comprising a hydrogel. Further provided herein are compositions further comprising a lipid nanoparticle. Further provided herein are compositions wherein the composition is in a liquid, semiliquid, or solid dosage form. Further provided herein are compositions wherein the composition is in the dosage form of a tablet, pill, capsule, lotion, lozenge, slurry, or cream.

Also provided herein are compositions, wherein the composition comprises: a nanoparticle carrier comprising a plurality of nanoparticles; and a polymer, wherein the polymer comprises a plurality of cannabinoid units, wherein the polymer comprises an ester linker, and wherein upon degradation of the ester linker a released cannabinoid is present in an amount sufficient for a phenolic antioxidant activity. Further provided here are compositions, wherein the nanoparticle carrier is at least one of: a liposome, nanoliposome, solid lipid nanoparticle (SLN), or a nanostructured lipid carrier (NLC). Further provided herein are compositions wherein the nanoparticles are between 100 to 300 nm in diameter. Further provided herein are compositions wherein the polymer does not comprise a free phenol moiety. Further provided herein are compositions wherein the composition, prior to degradation, does not provide for phenolic antioxidant activity. Further provided herein are compositions wherein upon degradation of the ester linker a released cannabinoid is present in an amount sufficient for modulating an immune response. Further provided herein are compositions wherein the degradation is by hydrolysis. Further provided herein are compositions wherein the polymer, when in a non-degraded state does not provide for the phenolic antioxidant activity. Further provided herein are compositions wherein the plurality of cannabinoid units comprises identical cannabinoid structures. Further provided herein are compositions wherein the plurality of cannabinoid units comprises different cannabinoid structures. Further provided herein are compositions wherein the plurality of cannabinoid units comprising the polymer are linked covalently by ester linkers. Further provided herein are compositions wherein the ester linker is selected from an adipoyl ester, adipoyl diester, a methyl ester, an ethyl ester, a hydrazone ester, a (poly) anhydride ester, or a combination thereof. Further provided here are compositions wherein the ester linker is adipoyl diester. Further provided herein are compositions wherein the plurality of cannabinoid units comprising the polymer are linked covalently by ether linkers. Further provided herein are compositions wherein the ether linker is selected from an alkyl ether, an alkenyl ether, a vinyl ether, or a combination thereof. Further provided herein are compositions wherein the polymer comprises an endcapping group. Further provided herein are compositions wherein the endcapping group is a linear or branched alcohol, or a cannabinoid unit having one hydroxyl group, acid group, or ester group before reaction with the polymer. Further provided herein are compositions wherein the plurality of cannabinoid units comprising the polymer are linked covalently by alternating ether and ester linkers. Further provided herein are compositions wherein the plurality of cannabinoid units are linked in a linear polymeric network. Further provided herein are compositions wherein the plurality of cannabinoid units is linked in a branched polymeric network. Further provided herein are compositions wherein upon degradation, a cannabinoid unit is released after 2 hours. Further provided here are compositions wherein the cannabinoid units comprise a cannabinoid described in TABLE 1. Further provided herein are compositions wherein each cannabinoid unit is independently derived from CBG, CBD, CBC, CBND, dihydro-DHCBD, CBD-V, CBC-V, CBND-C2, or dihydro-DHCBD-V, and wherein each cannabinoid unit is covalently bound to the linker at a phenolic oxygen. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise PLGA or PCL, or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the cannabinoid units comprise one or more of the cannabinoids listed in TABLE 1. Further provided herein are compositions wherein the composition further comprises a pH adjusting agent. Further provided herein are compositions wherein the pH adjusting agent is a silica particle. Further provided herein are compositions wherein the composition further comprises a second polymer. Further provided herein are compositions wherein the second polymer is a solid dispersion. Further provided herein are compositions wherein the second polymer is a selected from starch, polylactic acid, polyhydroxybutanoate, or a combination thereof. Further provided herein are compositions wherein the composition further comprises an active pharmaceutical ingredient (API) for co-administration, to a subject. Further provided herein are compositions wherein the API is administered to the subject in the same composition. Further provided herein are compositions wherein the API is administered to the subject in a second composition. Further provided herein are compositions wherein the composition further comprises a second, biodegradable polymer. Further provided herein are compositions wherein the biodegradable polymer is polylactic acid (PLA). Further provided herein are compositions wherein the polymers are blended. Further provided herein are compositions wherein the API is selected from TABLE 2 or is a combination of APIs listed in TABLE 2. Further provided herein are compositions wherein the polymers effect API release from the composition, as compared to a composition not comprising the polymers. Further provided herein are compositions wherein the composition comprising the polymer further comprises a bioactive polycannabinoid polymer, and wherein the bioactive polycannabinoid polymer has an average molecular weight of less than 50,000 daltons. Further provided herein are compositions further comprising a hydrogel. Further provided herein are compositions further comprising a lipid nanoparticle. Further provided herein are compositions wherein the composition is in a liquid, semiliquid, or solid dosage form. Further provided herein are compositions wherein the composition is in the dosage form of a tablet, pill, capsule, lotion, lozenge, slurry, or cream.

Provided herein are compositions, wherein the composition comprises: a polymer, wherein the polymer comprises a plurality of cannabinoid units, wherein the polymer comprises at least about 20 cannabinoid units, and wherein the polymer resists degradation at temperatures below about 150 degrees C.; and a carrier. Further provided here are compositions wherein the polymer degrades between 120 to 130 degrees C. Further provided here are compositions wherein the carrier is a solvent for the polymer. Further provided herein are compositions wherein the carrier is a lipid, optionally wherein the lipid is an oil at room temperature. Further provided herein are compositions wherein the oil is a food safe oil. Further provided herein are compositions wherein the oil is medium-chain triglyceride (MCT) oil, hemp seed oil, olive oil, or avocado oil, or a combination of any of the foregoing. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise PLGA or PCL, or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise PLGA or PCL, or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the cannabinoid units comprise one or more of the cannabinoids listed in TABLE 1. Further provided herein are compositions wherein the composition further comprises a pH adjusting agent. Further provided herein are compositions wherein the pH adjusting agent is a silica particle. Further provided herein are compositions wherein the composition further comprises a second polymer. Further provided herein are compositions wherein the second polymer is a solid dispersion. Further provided herein are compositions wherein the second polymer is a selected from starch, polylactic acid, polyhydroxybutanoate, or a combination thereof. Further provided herein are compositions wherein the composition further comprises an active pharmaceutical ingredient (API) for co-administration, to a subject. Further provided herein are compositions wherein the API is administered to the subject in the same composition. Further provided herein are compositions wherein the API is administered to the subject in a second composition. Further provided herein are compositions wherein the composition further comprises a second, biodegradable polymer. Further provided herein are compositions wherein the biodegradable polymer is polylactic acid (PLA). Further provided herein are compositions wherein the polymers are blended. Further provided herein are compositions wherein the API is selected from TABLE 2 or is a combination of APIs listed in TABLE 2. Further provided herein are compositions wherein the polymers effect API release from the composition, as compared to a composition not comprising the polymers. Further provided herein are compositions wherein the composition comprises the polymer further comprises a bioactive polycannabinoid polymer, and wherein the bioactive polycannabinoid polymer has an average molecular weight of less than 50,000 daltons. Further provided herein are compositions further comprising a hydrogel. Further provided herein are compositions further comprising a lipid nanoparticle. Further provided herein are compositions wherein the composition is in a liquid, semiliquid, or solid dosage form. Further provided herein are compositions wherein the composition is in the dosage form of a tablet, pill, capsule, lotion, lozenge, slurry, or cream.

Provided herein are compositions, wherein the composition comprises: a polymer, wherein the polymer comprises a plurality of cannabinoid units, and a terpene, or derivative thereof. Further provided here are compositions wherein the terpene is at least one of: D-limonene, geraniol, humulene, linalool, myrcene, terpineol, terpinolene, α-pinene, and β-caryophyllene. Further provided here are compositions wherein the polymer, when in a non-degraded state does not provide for the phenolic antioxidant activity. Further provided herein are compositions wherein the plurality of cannabinoid units comprises identical cannabinoid structures. Further provided herein are compositions wherein the plurality of cannabinoid units comprises different cannabinoid structures. Further provided herein are compositions wherein the plurality of cannabinoid units comprising the polymer are linked covalently by ester linkers. Further provided herein are compositions wherein the ester linker is selected from an adipoyl ester, adipoyl diester, a methyl ester, an ethyl ester, a hydrazone ester, a (poly) anhydride ester, or a combination thereof. Further provided herein are compositions wherein the ester linker is adipoyl diester. Further provided herein are compositions wherein the plurality of cannabinoid units comprising the polymer are linked covalently by ether linkers. Further provided herein are compositions wherein the ether linker is selected from an alkyl ether, an alkenyl ether, a vinyl ether, or a combination thereof. Further provided herein are compositions wherein the polymer comprises an endcapping group. Further provided herein are compositions wherein the endcapping group is a linear or branched alcohol, or a cannabinoid unit having one hydroxyl group, acid group, or ester group before reaction with the polymer. Further provided herein are compositions wherein the plurality of cannabinoid units comprising the polymer are linked covalently by alternating ether and ester linkers. Further provided herein are compositions wherein the plurality of cannabinoid units are linked in a linear polymeric network. Further provided herein are compositions wherein the plurality of cannabinoid units is linked in a branched polymeric network. Further provided herein are compositions wherein a terminal cannabinoid unit comprises a different cannabinoid unit than an internal cannabinoid unit. Further provided herein are compositions wherein the terminal cannabinoid unit is released in an amount higher than an internal cannabinoid unit. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise PLGA or PCL, or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the cannabinoid units comprise one or more of the cannabinoids listed in TABLE 1. Further provided herein are compositions wherein the composition further comprises a pH adjusting agent. Further provided herein are compositions wherein the pH adjusting agent is a silica particle. Further provided herein are compositions wherein the composition further comprises a second polymer. Further provided herein are compositions wherein the second polymer is a solid dispersion. Further provided herein are compositions wherein the second polymer is a selected from starch, polylactic acid, polyhydroxybutanoate, or a combination thereof. Further provided herein are compositions wherein the composition further comprises an active pharmaceutical ingredient (API) for co-administration, to a subject. Further provided herein are compositions wherein the API is administered to the subject in the same composition. Further provided herein are compositions wherein the API is administered to the subject in a second composition. Further provided herein are compositions wherein the composition further comprises a second, biodegradable polymer. Further provided herein are compositions wherein the biodegradable polymer is polylactic acid (PLA). Further provided herein are compositions wherein the polymers are blended. Further provided herein are compositions wherein the API is selected from TABLE 2 or is a combination of APIs listed in TABLE 2. Further provided herein are compositions wherein the polymers effect API release from the composition, as compared to a composition not comprising the polymers. Further provided herein are compositions wherein the composition comprises the polymer further comprises a bioactive polycannabinoid polymer, and wherein the bioactive polycannabinoid polymer has an average molecular weight of less than 50,000 daltons. Further provided herein are compositions further comprising a hydrogel. Further provided herein are compositions further comprising a lipid nanoparticle. Further provided herein are compositions wherein the composition is in a liquid, semiliquid, or solid dosage form. Further provided herein are compositions wherein the composition is in the dosage form of a tablet, pill, capsule, lotion, lozenge, slurry, or cream.

Provided herein are compositions, wherein the composition comprises: a polymer comprising a plurality of cannabinoid units, wherein the plurality of cannabinoid units comprises at least about 50 different cannabinoids, or derivatives thereof; and a lipid. Further provided herein are compositions wherein the lipid is in a liquid phase at 25 degrees C. Further provided herein are compositions wherein the lipid is in a solid phase at 25 degrees C. Further provided herein are compositions wherein the lipid is in a semi solid phase at 25 degrees C. Further provided herein are compositions wherein the composition further comprises a delivery system. Further provided herein are compositions wherein the delivery system comprises a lipid. Further provided herein are compositions wherein the delivery system comprises polymeric microparticles. Further provided herein are compositions wherein the polymeric microparticles comprise PLGA or PCL, or a combination thereof. Further provided herein are compositions wherein the delivery system comprises an inclusion complex. Further provided herein are compositions wherein the delivery system comprises vesicles or emulsions, or a combination thereof. Further provided herein are compositions wherein the composition further comprises an active pharmaceutical ingredient (API) for co-administration, to a subject. Further provided herein are compositions wherein the API is administered to the subject in the same composition. Further provided herein are compositions wherein the API is administered to the subject in a second composition. Further provided herein are compositions wherein the composition further comprises a second, biodegradable polymer. Further provided herein are compositions wherein the biodegradable polymer is polylactic acid (PLA). Further provided herein are compositions wherein the polymers are blended. Further provided herein are compositions wherein the API is selected from TABLE 2 or is a combination of APIs listed in TABLE 2. Further provided herein are compositions wherein the polymers effect API release from the composition, as compared to a composition not comprising the polymers. Further provided herein are compositions wherein the composition comprises the polymer further comprises a bioactive polycannabinoid polymer, and wherein the bioactive polycannabinoid polymer has an average molecular weight of less than 50,000 daltons. Further provided herein are compositions further comprising a hydrogel. Further provided herein are compositions further comprising a lipid nanoparticle. Further provided herein are compositions wherein the composition is in a liquid, semiliquid, or solid dosage form. Further provided herein are compositions wherein the composition is in the dosage form of a tablet, pill, capsule, lotion, lozenge, slurry, or cream.

Also provided herein are methods for extracting the cannabinoid polymer from a composition described herein, wherein the method comprises, heating to 100 degrees C., and allowing the heated composition to cool.

Also provided herein are pharmaceutical compositions comprising a composition described herein, and a pharmaceutically acceptable excipient.

Also provided herein are methods for delivery of a cannabinoid, wherein the method comprises administering to a subject a composition or a pharmaceutical composition described herein. Further provided herein are methods wherein the administering is transdermal, oral, or intravenous. Also provided herein are methods for reduction of anxiety, comprising administering to a subject a composition described herein. Provided herein are methods for reduction of sleep disturbance, comprising administering to a subject a composition described herein. Provided herein are methods for treatment of cancer, comprising administering to a subject a composition described herein. Further provided herein are methods wherein the cancer is breast cancer. Further provided herein are methods wherein the breast cancer is triple negative breast cancer. Further provided herein are methods wherein the breast cancer is advanced stage breast cancer. Further provided herein are methods wherein the subject receives the chemotherapy or radiation therapy prior, concurrently with, or following administration of the composition or the pharmaceutical composition. Further provided herein are methods wherein the subject is administered naltrexone. Further provided herein are methods wherein the subject is administered antidepressants, anti-anxiety medication, or cancer therapy.

### BRIEF DESCRIPTION OF THE DRAWINGS

FIG. 1 is a schematic representation of a branched cannabinoid polymer under acidic or basic conditions resulting in the release of a singular cannabinoid molecule.

FIG. 2 is a schematic representation of a branched cannabinoid polymer under acidic or basic conditions resulting in the release of a singular cannabinoid molecule (or other previously covalently bound API), where more than one species of cannabinoid is represented.

FIG. 3. is a schematic representation of an unbranched cannabinoid polymer under acidic or basic conditions resulting in the release of a singular cannabinoid molecule.

FIG. 4. is a schematic representation of an unbranched cannabinoid polymer under acidic or basic conditions resulting in the release of a singular cannabinoid molecule (or other previously covalently bound API), where more than one species of cannabinoid is represented.

FIG. 5 is a schematic representation of a daisy-chain type cannabinoid polymer under acidic or basic conditions resulting in the release of a singular cannabinoid molecule.

FIG. 6 is a schematic representation of a daisy-chain type cannabinoid polymer under acidic or basic conditions resulting in the release of a singular cannabinoid molecule (or other previously covalently bound API), where more than one species of cannabinoid is represented.

FIG. 7 depicts an exemplary schematic representation of a polycannabidiol (polyCBD) nanoparticle and the release of a cannabidiol (CBD) monomer with the addition of water (H₂O) in conjunction with a diffusion process.

### DETAILED DESCRIPTION

Provided herein are compositions, kits, devices and uses thereof for treatment of various conditions. Briefly, further described herein are cannabinoid and cannabinoid analogues agonists, cannabinoid polymers, compositions comprising cannabinoid polymers, cannabinoid polymer composition carrier systems; conditions for use of such compositions, and forms of administration.

### Definitions

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, the term “about” or “approximately” means a range of up to ±20%, of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The term “effective amount” or “therapeutically effective amount” refers to an amount that is sufficient to achieve or at least partially achieve the desired effect.

The term “effective” is used to describe an amount of a compound, composition or component which, when used within the context of its intended use, effects an intended result. The term effective subsumes all other effective amount or effective concentration terms, which are otherwise described or used in the present application.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically or cosmetically acceptable salts and derivatives, including prodrug and/or deuterated forms thereof where applicable, in context. Also included are deuterated compounds, in which one or more of hydrogen atoms in a compound described herein have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented or understood within the context of the compound shown and well-known rules for valence interactions.

As used herein, “alkyl” includes straight chain, branched, and cyclic saturated aliphatic hydrocarbon groups, having the specified number of carbon atoms, generally from 1 to about 20 carbon atoms, greater than 3 for the cyclic. Alkyl groups described herein typically have from 1 to about 20, specifically 3 to about 18, and more specifically about 6 to about 12 carbons atoms. Examples of alkyl include, but are not limited to, methyl, ethyl, n-propyl, isopropyl, n-butyl, 3-methylbutyl, t-butyl, n-pentyl, and sec-pentyl. As used herein, “cycloalkyl” indicates a monocyclic or multicyclic saturated or unsaturated hydrocarbon ring group, having the specified number of carbon atoms, usually from 3 to about 10 ring carbon atoms. Monocyclic cycloalkyl groups typically have from 3 to about 8 carbon ring atoms or from 3 to about 7 carbon ring atoms. Multicyclic cycloalkyl groups may have 2 or 3 fused cycloalkyl rings or contain bridged or caged cycloalkyl groups. Examples of cycloalkyl groups include cyclopropyl, cyclobutyl, cyclopentyl, or cyclohexyl as well as bridged or caged saturated ring groups such as norbornane or adamantane.

The term “heterocycloalkyl” is used to indicate saturated cyclic groups containing from 1 to about 3 heteroatoms chosen from N, O, and S, with remaining ring atoms being carbon. Heterocycloalkyl groups have from 3 to about 8 ring atoms, and more typically have from 5 to 7 ring atoms. A C₂-C₇ heterocycloalkyl group contains from 2 to about 7 carbon ring atoms and at least one ring atom chosen from N, O, and S. Examples of heterocycloalkyl groups include morpholinyl, piperazinyl, piperidinyl, and pyrrolidinyl groups.

As used herein, “heteroaryl” indicates a stable 5- to 7-membered monocyclic or 7- to 10-membered bicyclic heterocyclic ring which contains at least 1 aromatic ring that contains from 1 to 4, or specifically from 1 to 3, heteroatoms chosen from N, O, and S, with remaining ring atoms being carbon. When the total number of S and O atoms in the heteroaryl group exceeds 1, these heteroatoms are not adjacent to one another. In a specific embodiment, the total number of S and O atoms in the heteroaryl group is not more than 2. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, and 5,6,7,8-tetrahydroisoquinoline.

The term “patient” or “subject” is used throughout the specification to describe an animal, specifically a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

### Cannabinoids

Cannabis sativa L. (Cannabaceae) (aka “hemp”) is a plant cultivated industrially for its fiber content and nutrient rich seeds. As a crop, hemp produces a fiber content more than 250% higher than conventional fiber crops, while requiring fewer chemical herbicides, fungicides, and pesticides. Cannabinoids are compounds found in the cannabis plant or synthetic compounds that can interact with the endocannabinoid system. Cannabinoids are lipophilic, and potentially acid-labile compounds. Because of their hydrophobic nature, cannabinoids are poorly absorbed systemically because of the poor dissolution of cannabinoids in the aqueous environment of gastrointestinal tract. As such, many forms of cannabinoids exhibit low bioavailability. In addition, cannabinoids have relatively low thermal stability, such as 130 degrees C. for decomposition, making the administration and prolonged storage of cannabinoids in a number of forms difficult to achieve on a sustained basis. Exemplary cannabinoids are listed, but not limited to those described in TABLE 1.

TABLE 1
Exemplary cannabinoid structures
Substructure: Diol (no salicylic)

| Compound | | |
|---|---|---|
| No. | Name (Abbreviation) | Chemical Structure |
| 1 | 4′-(3-Carboxyproply)- CBD | 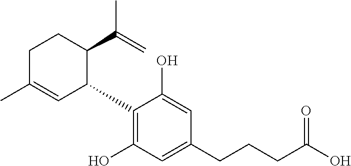 [SMILES: C=C(C)[C@@H]1CCC(C)=C[C@H]1c1c(O)cc(CCCC(=O)O)cc1O] |
| | | 4′-(3-Carboxyproply)-CBD |
| 2 | Cannabidiol (CBD) | 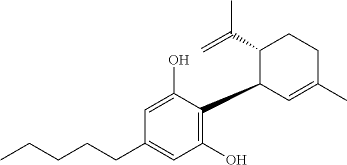 [SMILES: C=C(C)[C@@H]1CCC(C)=C[C@H]1c1c(O)cc(CCCCC)cc1O] |
| 3 | Cannabidivarin (CBDV) | 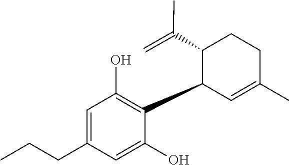 [SMILES: C=C(C)[C@@H]1CCC(C)=C[C@H]1c1c(O)cc(CCC)cc1O] |
| 4 | Cannabidiorcol (CBD-C1) | 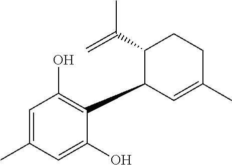 |
| 5 | Cannabielsoin (CBE-C5) | 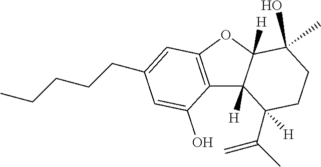 [SMILES: C=C(C)[C@@H]1CC[C@](C)(O)[C@H]2Oc3cc(CCCCC)cc(O)c3[C@@H]12] |
| 6 | Cannabielsoin (CBE-C3) | 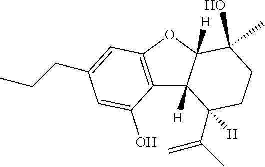 [SMILES: C=C(C)[C@@H]1CC[C@](C)(O)[C@H]2Oc3cc(CCC)cc(O)c3[C@@H]12] |
| 7 | Cannabiglendol-C3 (OH-iso-HHCV-C3) | 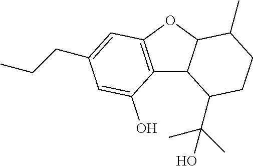 [SMILES: CCCc1cc(O)c2c(c1)OC1C(C)CCC(C(C)(C)O)C21] |
| 8 | RR-trans-10-O-Ethyl- cannabitriol (RR-trans-CBT-OEt- C5) | 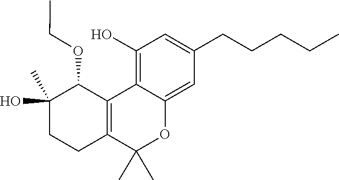 [SMILES: CCCCCc1cc(O)c2c(c1)OC(C)(C)C1=C2[C@@H](OCC)[C@](C)(O)CC1] |
| 9 | Cannabigerol (CBG) | 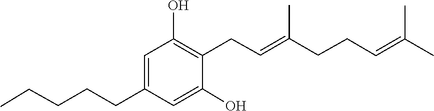 [SMILES: CCCCCc1cc(O)c(C/C=C(\C)CCC=C(C)C)c(O)c1] |
| 10 | Cannabigerovarin (CBGV) | 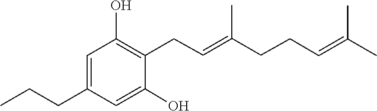 [SMILES: CCCc1cc(O)c(C/C=C(\C)CCC=C(C)C)c(O)c1] |
| 11 | Cannabinodiol (CBND) | 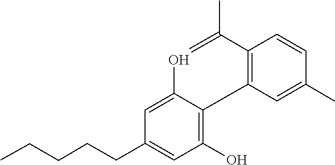 [SMILES: C=C(C)c1ccc(C)cc1-c1c(O)cc(CCCCC)cc1O] |
| 12 | Cannabinodiol-C2 (CBND-C2) | 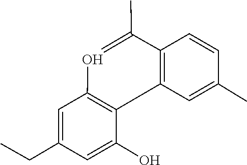 [SMILES: C=C(C)c1ccc(C)cc1-c1c(O)cc(CC)cc1O] |
| 13 | Cannabinodivarin (CBVD) | 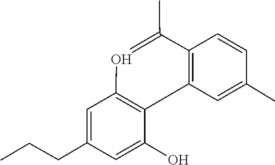 [SMILES: C=C(C)c1ccc(C)cc1-c1c(O)cc(CCC)cc1O] |
| 14 | Machaeridiol A | 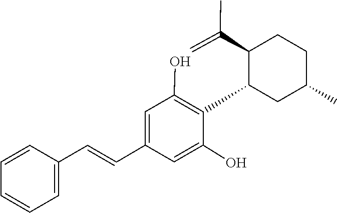 [SMILES: C=C(C)[C@H]1CC[C@H](C)C[C@@H]1c1c(O)cc(/C=C/c2ccccc2)cc1O] |
| 15 | Machaeridiol C | 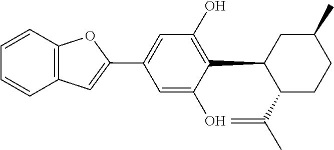 [SMILES: C=C(C)[C@H]1CC[C@H](C)C[C@@H]1c1c(O)cc(-c2cc3ccccc3o2)cc1O] |
| 16 | (+)-CBD | 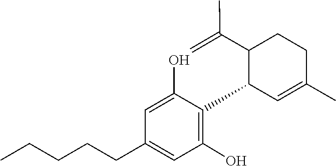 [SMILES: C=C(C)C1CCC(C)=C[C@@H]1c1c(O)cc(CCCCC)cc1O] |
| 17 | H2-CBD | 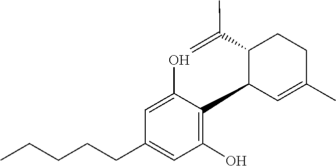 [SMILES: C=C(C)[C@@H]1CCC(C)=C[C@H]1c1c(O)cc(CCCCC)cc1O] |
| 18 | H4-CBD | 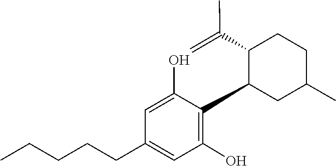 [SMILES: C=C(C)[C@@H]1CCC(C)C[C@H]1c1c(O)cc(CCCCC)cc1O] |
| 19 | (−)-DMH-CBD | 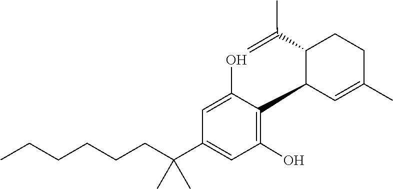 [SMILES: C=C(C)[C@@H]1CCC(C)=C[C@H]1c1c(O)cc(C(C)(C)CCCCCC)cc1O] |
| 20 | H2-DMH-CBD | 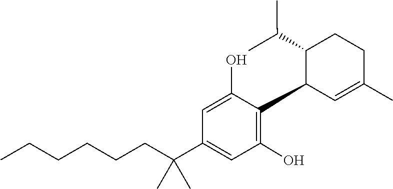 [SMILES: CCCCCCC(C)(C)c1cc(O)c([C@@H]2C=C(C)CC[C@H]2C(C)C)c(O)c1] |
| 21 | H4-DMH-CBD | 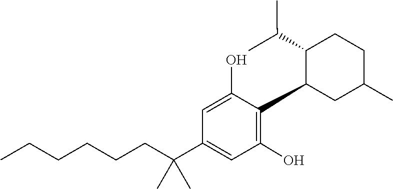 [SMILES: CCCCCCC(C)(C)c1cc(O)c([C@@H]2CC(C)CC[C@H]2C(C)C)c(O)c1] |
| 22 | KLS-13019 | 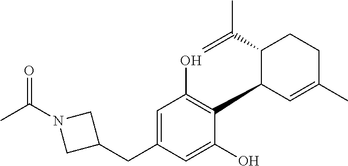 [SMILES: C=C(C)[C@@H]1CCC(C)=C[C@H]1c1c(O)cc(CC2CN(C(C)=O)C2)cc1O] |
| 23 | 4′-[2-(1H-1,2,3- Triazol-yl)ethyl]-CBD | 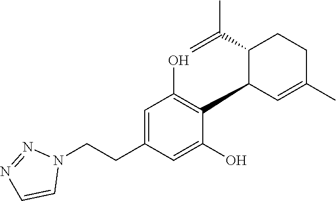 [SMILES: C=C(C)[C@@H]1CCC(C)=C[C@H]1c1c(O)cc(CCn2ccnn2)cc1O] |
| 24 | 4′-(2- Morpholinoethyl)-CBD | 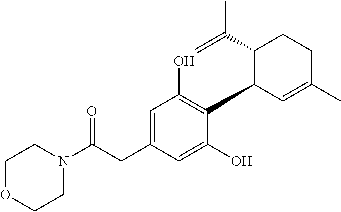 [SMILES: C=C(C)[C@@H]1CCC(C)=C[C@H]1c1c(O)cc(CC(=O)N2CCOCC2)cc1O] |
| 25 | 4′-(2-Ethoxyethyl)- CBD | 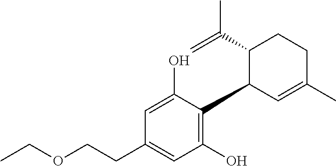 [SMILES: C=C(C)[C@@H]1CCC(C)=C[C@H]1c1c(O)cc(CCOCC)cc1O] |
| 26 | 3′-Cl-CBD | 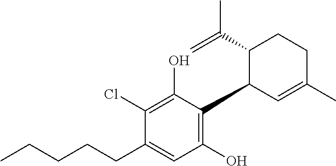 [SMILES: C=C(C)[C@@H]1CCC(C)=C[C@H]1c1c(O)cc(CCCCC)c(Cl)c1O] |
| 27 | 3′,5′-diCl-CBD | 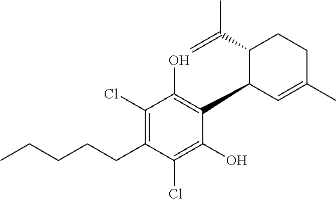 [SMILES: C=C(C)[C@@H]1CCC(C)=C[C@H]1c1c(O)c(Cl)c(CCCCC)c(Cl)c1O] |
| 28 | 3′-Br-CBD | 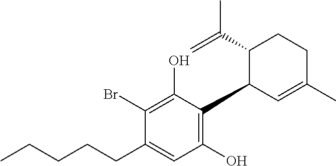 [SMILES: C=C(C)[C@@H]1CCC(C)=C[C@H]1c1c(O)cc(CCCCC)c(Br)c1O] |
| 29 | 3′,5′-diBr-CBD | 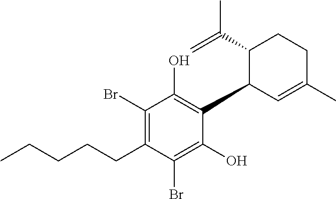 [SMILES: C=C(C)[C@@H]1CCC(C)=C[C@H]1c1c(O)c(Br)c(CCCCC)c(Br)c1O] |
| 30 | 3′,5′-diI-CBD | 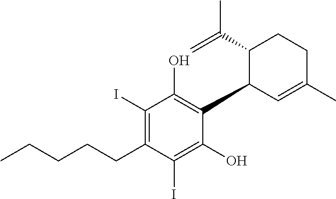 [SMILES: C=C(C)[C@@H]1CCC(C)=C[C@H]1c1c(O)c(I)c(CCCCC)c(I)c1O] |
| 31 | 3′-I-CBD | 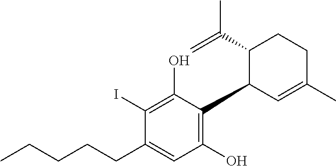 [SMILES: C=C(C)[C@@H]1CCC(C)=C[C@H]1c1c(O)cc(CCCCC)c(I)c1O] |
| 32 | HUF-101 | 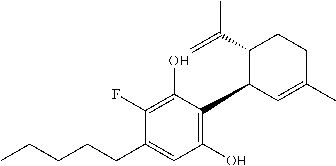 [SMILES: C=C(C)[C@@H]1CCC(C)=C[C@H]1c1c(O)cc(CCCCC)c(F)c1O] |
| 33 | HUF-103 | 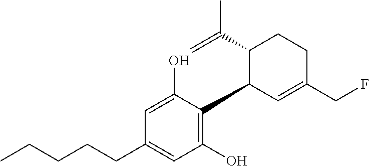 [SMILES: C=C(C)[C@@H]1CCC(CF)=C[C@H]1c1c(O)cc(CCCCC)cc1O] |
| 34 | O-1602 | 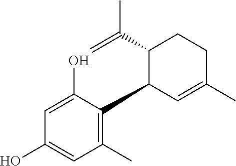 [SMILES: C=C(C)[C@@H]1CCC(C)=C[C@H]1c1c(C)cc(O)cc1O] |
| 35 | Abn-CBD | 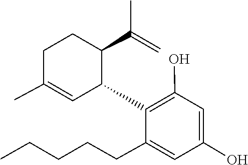 [SMILES: C=C(C)[C@@H]1CCC(C)=C[C@H]1c1c(O)cc(O)cc1CCCCC] |
| 36 | 7-COOH-CBD | 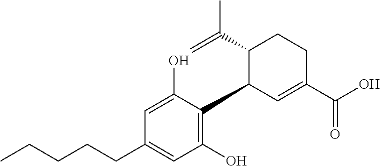 [SMILES: C=C(C)[C@@H]1CCC(C(=O)O)=C[C@H]1c1c(O)cc(CCCCC)cc1O] |

Substructure: Salicylic acid

| | | |
|---|---|---|
| 37 | Cannabichromenic Acid (CBCA) | 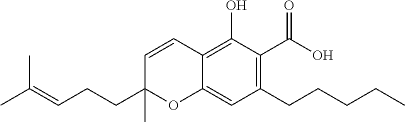 [SMILES: CCCCCc1cc2c(c(O)c1C(=O)O)C=CC(C)(CCC=C(C)C)O2] |
| 38 | Cannabichromevarinic Acid (CBCVA) | 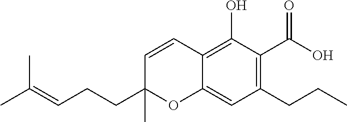 [SMILES: CCCc1cc2c(c(O)c1C(=O)O)C=CC(C)(CCC=C(C)C)O2] |
| 39 | Cannabicyclolic Acid (CBLA) | 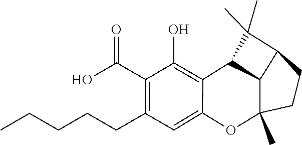 [SMILES: CCCCCc1cc2c(c(O)c1C(=O)O)[C@H]1[C@H]3[C@H](CC[C@]3(C)O2)C1(C)C] |
| 40 | Cannabicyclovarinic Acid (CBLVA) | 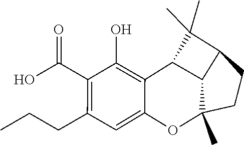 [SMILES: CCCc1cc2c(c(O)c1C(=O)O)[C@H]1[C@@H]3[C@H](CC[C@]3(C)O2)C1(C)C] |
| 41 | Cannabidiolic acid monomethyl ether (CBDAM) | 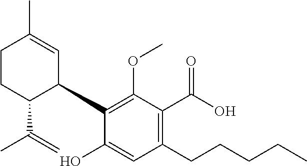 [SMILES: C=C(C)[C@@H]1CCC(C)=C[C@H]1c1c(O)cc(CCCCC)c(C(=O)O)c1OC] |
| 42 | Cannabigerolic Acid Monomethyl ether (CBGAM) | 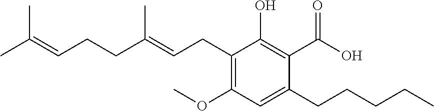 [SMILES: CCCCCc1cc(OC)c(C/C=C(\C)CCC=C(C)C)c(O)c1C(=O)O] |
| 43 | Cannabinolic Acid (CBNA) | 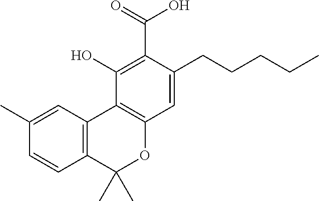 [SMILES: CCCCCc1cc2c(c(O)c1C(=O)O)-c1cc(C)ccc1C(C)(C)O2] |
| 44 | Δ9-Tetrahydro- cannabinolic acid A (Δ9-THCA-C5 A) | 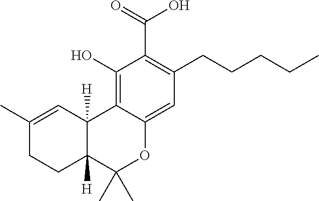 [SMILES: CCCCCc1cc2c(c(O)c1C(=O)O)[C@@H]1C=C(C)CC[C@H]1C(C)(C)O2] |
| 45 | Δ9-Tetrahydro- cannabinolic acid B (Δ9-THCA-C5 B) | 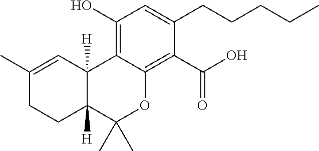 [SMILES: CCCCCc1cc(O)c2c(c1C(=O)O)OC(C)(C)[C@@H]1CCC(C)=C[C@@H]21] |
| 46 | Δ9-Tetrahydro- cannabinolic acid-C4A (Δ9-THCA-C4A) | 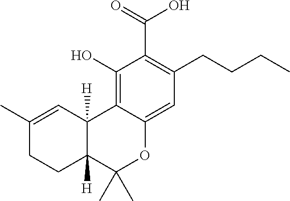 [SMILES: CCCCc1cc2c(c(O)c1C(=O)O)[C@@H]1C=C(C)CC[C@H]1C(C)(C)O2] |
| 47 | Δ9-Tetrahydro- cannabinolic acid-C4B (Δ9-THCA-C4B) | 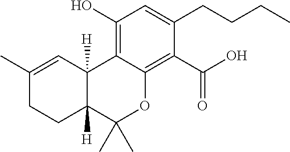 [SMILES: CCCCc1cc(O)c2c(c1C(=O)O)OC(C)(C)[C@@H]1CCC(C)=C[C@@H]21] |
| 48 | Δ9-Tetrahydro- cannabivarinic acid A (Δ9-THCVA-C3A) | 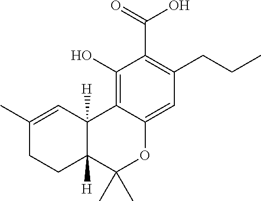 [SMILES: CCCc1cc2c(c(O)c1C(=O)O)[C@@H]1C=C(C)CC[C@H]1C(C)(C)O2] |
| 49 | Δ9-Tetrahydro- cannabiorcolic acid A (Δ9-THCOA-C1A) | 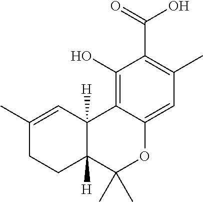 |
| 50 | Δ9-Tetrahydro- cannabiorcolic acid B (Δ9-THCOA-C1B) | 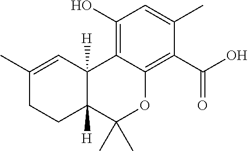 [SMILES: CC1=C[C@H]2c3c(O)cc(C)c(C(=O)O)c3OC(C)(C)[C@@H]2CC1] |
| 51 | (−)-Δ8-trans- (6aR,10aR)- Tetrahydrocannabinolic acid A (Δ8-THCA-C5 A) | 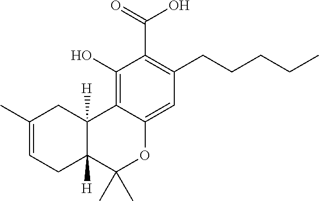 [SMILES: CCCCCc1cc2c(c(O)c1C(=O)O)[C@@H]1CC(C)=CC[C@H]1C(C)(C)O2] |
| 52 | HU-410 | 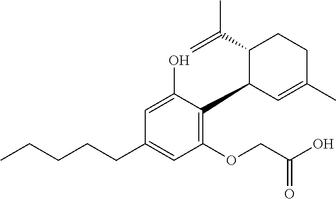 [SMILES: C=C(C)[C@@H]1CCC(C)=C[C@H]1c1c(O)cc(CCCCC)cc1OCC(=O)O] |

Substructure: Diol and Salicylic Acid

| | | |
|---|---|---|
| 53 | Cannabidiolic Acid (CBDA) | 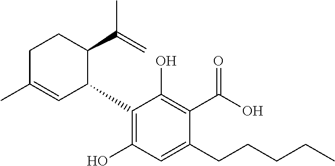 [SMILES: C=C(C)[C@@H]1CCC(C)=C[C@H]1c1c(O)cc(CCCCC)c(C(=O)O)c1O] |
| 54 | Cannabidivarininc Acid (CBDVA) | 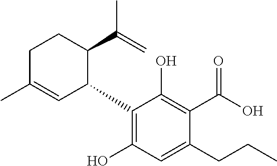 [SMILES: C=C(C)[C@@H]1CCC(C)=C[C@H]1c1c(O)cc(CCC)c(C(=O)O)c1O] |
| 55 | Cannabidiorcolic Acid (CBDA-C1) | 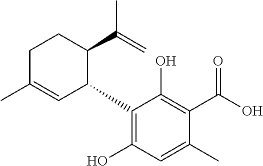 [SMILES: C=C(C)[C@@H]1CCC(C)=C[C@H]1c1c(O)cc(C)c(C(=O)O)c1O] |
| 56 | Cannabielsoic acid B (CBEA-C5 B) | 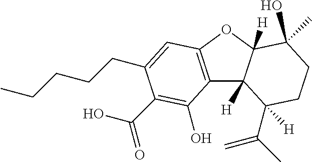 [SMILES: C=C(C)[C@@H]1CC[C@](C)(O)[C@H]2Oc3cc(CCCCC)c(C(=O)O)c(O)c3[C@@H]12] |
| 57 | Cannabielsoin acid A (CBEA-C5 A) | 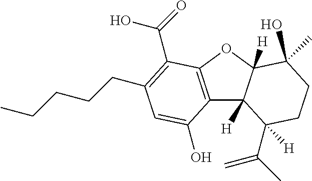 [SMILES: C=C(C)[C@@H]1CC[C@](C)(O)[C@H]2Oc3c(C(=O)O)c(CCCCC)cc(O)c3[C@@H]12] |
| 58 | Cannabielsoic acid B (CBEA-C3 B) | 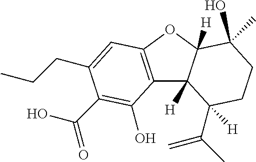 [SMILES: C=C(C)[C@@H]1CC[C@](C)(O)[C@H]2Oc3cc(CCC)c(C(=O)O)c(O)c3[C@@H]12] |
| 59 | (E)-Cannabigerolic Acid ((E)-CBGA) | 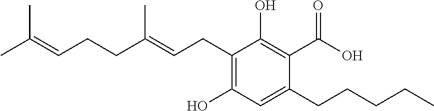 [SMILES: CCCCCc1cc(O)c(C/C=C(\C)CCC=C(C)C)c(O)c1C(=O)O] |
| 60 | (Z)-Cannabigerolic Acid ((Z)-CBGA) | 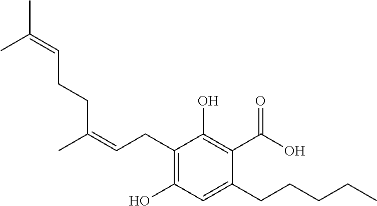 [SMILES: CCCCCc1cc(O)c(C/C=C(/C)CCC=C(C)C)c(O)c1C(=O)O] |
| 61 | Cannabigerovarinic Acid (CBGVA) | 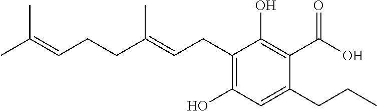 [SMILES: CCCc1cc(O)c(C/C=C(\C)CCC=C(C)C)c(O)c1C(=O)O] |
| 62 | HU-331 | 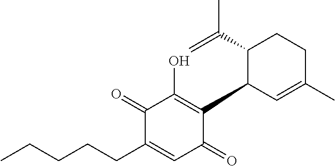 [SMILES: C=C(C)[C@@H]1CCC(C)=C[C@H]1C1=C(O)C(=O)C(CCCCC)=CC1=O] |
| 63 | HU-427 | 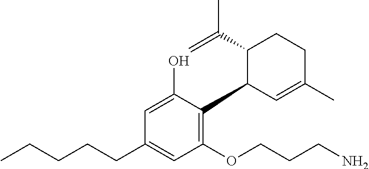 [SMILES: C=C(C)[C@@H]1CCC(C)=C[C@H]1c1c(O)cc(CCCCC)cc1OCCCN] |
| 64 | HU-308 | 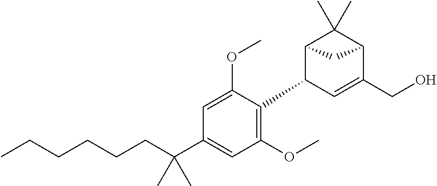 [SMILES: CCCCCCC(C)(C)c1cc(OC)c([C@H]2C=C(CO)[C@H]3C[C@@H]2C3(C)C)c(OC)c1] |
| 65 | HU-433 | 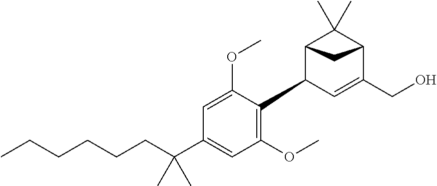 [SMILES: CCCCCCC(C)(C)c1cc(OC)c([C@@H]2C=C(CO)[C@@H]3C[C@H]2C3(C)C)c(OC)c1] |
| 66 | Cannabichromene (CBC) | 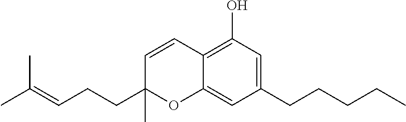 [SMILES: CCCCCc1cc(O)c2c(c1)OC(C)(CCC=C(C)C)C=C2] |
| 67 | Cannabichromevarin (CBCV) | 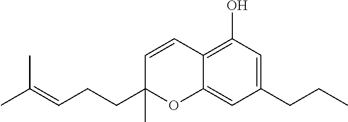 [SMILES: CCCc1cc(O)c2c(c1)OC(C)(CCC=C(C)C)C=C2] |
| 68 | Cannabicyclol (CBL) | 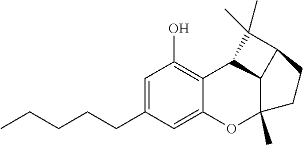 [SMILES: CCCCCc1cc(O)c2c(c1)O[C@@]1(C)CC[C@H]3[C@@H]1[C@H]2C3(C)C] |
| 69 | Cannabicyclovarin (CBLV) | 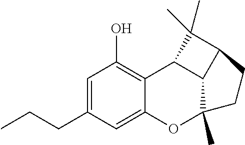 [SMILES: CCCc1cc(O)c2c(c1)O[C@@]1(C)CC[C@H]3[C@H]1[C@H]2C3(C)C] |
| 70 | Cannabidiol monomethyl ether (CBDM) | 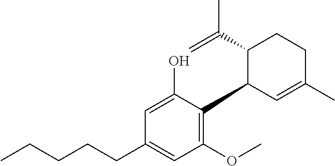 [SMILES: C=C(C)[C@@H]1CCC(C)=C[C@H]1c1c(O)cc(CCCCC)cc1OC] |
| 71 | Dehydrocannabifuran (DCBF-C5) | 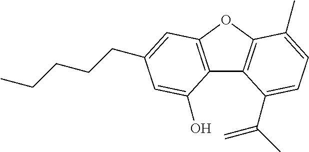 [SMILES: C=C(C)c1ccc(C)c2oc3cc(CCCCC)cc(O)c3c12] |
| 72 | Cannabifuran (CBF-C5) | 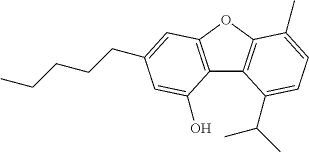 [SMILES: CCCCCc1cc(O)c2c(c1)oc1c(C)ccc(C(C)C)c12] |
| 73 | Trans- Isotetrahydrocannabinol | 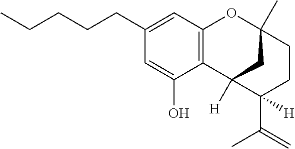 [SMILES: C=C(C)[C@@H]1CC[C@]2(C)C[C@H]1c1c(O)cc(CCCCC)cc1O2] |
| 74 | Cis- Isotetrahydrocannabivarin | 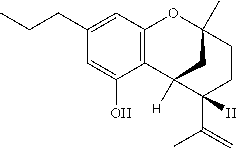 [SMILES: C=C(C)[C@H]1CC[C@]2(C)C[C@H]1c1c(O)cc(CCC)cc1O2] |
| 75 | Trans- Isotetrahydrocannabivarin | 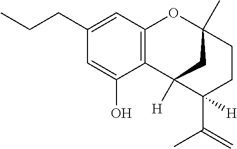 [SMILES: C=C(C)[C@@H]1CC[C@]2(C)C[C@H]1c1c(O)cc(CCC)cc1O2] |
| 76 | Cannabichromanone (CBCN-C5) | 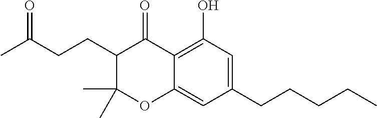 [SMILES: CCCCCc1cc(O)c2c(c1)OC(C)(C)C(CCC(C)=O)C2=O] |
| 77 | Cannabichromanone- C3 (CBCN-C3) | 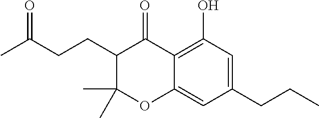 [SMILES: CCCc1cc(O)c2c(c1)OC(C)(C)C(CCC(C)=O)C2=O] |
| 78 | 10-Oxo-Δ6a(10a)- tetrahydrocannabinol (OTHC) | 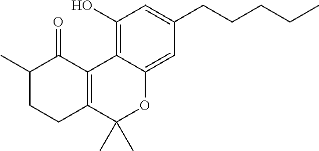 [SMILES: CCCCCc1cc(O)c2c(c1)OC(C)(C)C1=C2C(=O)C(C)CC1] |
| 79 | Cannabigerol Monomethyl ether (CBGM) | 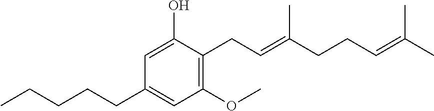 [SMILES: CCCCCc1cc(O)c(C/C=C(\C)CCC=C(C)C)c(OC)c1] |
| 80 | Cannabinol (CBN) | 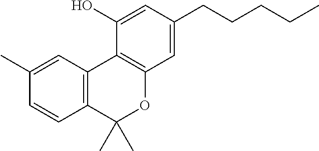 [SMILES: CCCCCc1cc(O)c2c(c1)OC(C)(C)c1ccc(C)cc1-2] |
| 81 | Cannabiorcol (CBN-C1) | 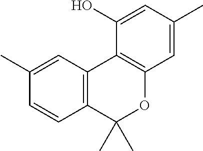 |
| 82 | Cannabinol-C2 (CBN-C2) | 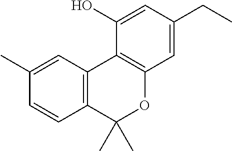 [SMILES: CCc1cc(O)c2c(c1)OC(C)(C)c1ccc(C)cc1-2] |
| 83 | Cannabivarin (CBV) | 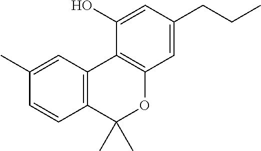 [SMILES: CCCc1cc(O)c2c(c1)OC(C)(C)c1ccc(C)cc1-2] |
| 84 | Cannabinol-C4 (CBN-C4) | 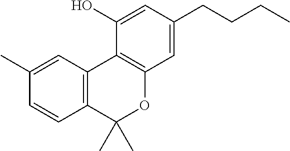 [SMILES: CCCCc1cc(O)c2c(c1)OC(C)(C)c1ccc(C)cc1-2] |
| 85 | Δ9- Tetrahydrocannabinol (Δ9-THC-C5) | 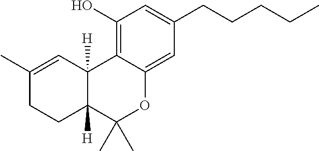 [SMILES: CCCCCc1cc(O)c2c(c1)OC(C)(C)[C@@H]1CCC(C)=C[C@@H]21] |
| 86 | Δ9- Tetrahydrocannabinol- C4 (Δ9-THC-C4) | 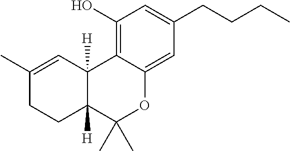 [SMILES: CCCCc1cc(O)c2c(c1)OC(C)(C)[C@@H]1CCC(C)=C[C@@H]21] |
| 87 | Δ9- Tetrahydrocannabivarin (Δ9-THCV-C3) | 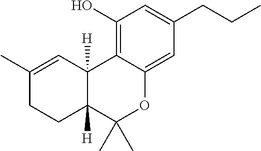 [SMILES: CCCc1cc(O)c2c(c1)OC(C)(C)[C@@H]1CCC(C)=C[C@@H]21] |
| 88 | Δ9- Tetrahydrocannabiorcol (Δ9-THCO-C1) | 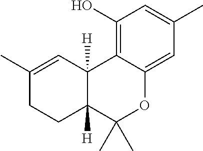 |
| 89 | (−)-Δ8-trans- (6aR,10aR)-Δ8- Tetrahydrocannabinol (Δ8-THC-C5) | 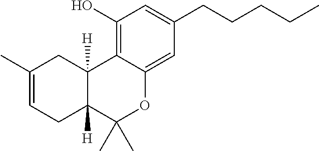 [SMILES: CCCCCc1cc(O)c2c(c1)OC(C)(C)[C@@H]1CC=C(C)C[C@@H]21] |
| 90 | (−)-(6aS,10aR)-Δ9- Tetrahydrocannabinol ((−)-cis-Δ9-THC-C5) | 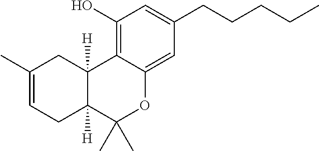 [SMILES: CCCCCc1cc(O)c2c(c1)OC(C)(C)[C@H]1CC=C(C)C[C@@H]21] |
| 91 | Desoxy-CBD | 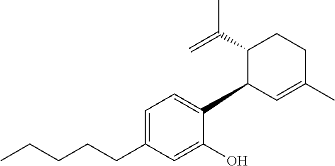 [SMILES: C=C(C)[C@@H]1CCC(C)=C[C@H]1c1ccc(CCCCC)cc1O] |
| 92 | CBD-Q (VIII) | 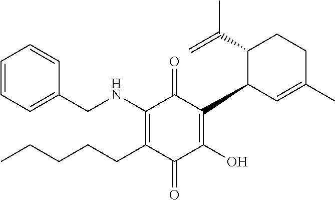 [SMILES: C=C(C)[C@@H]1CCC(C)=C[C@H]1C1=C(O)C(=O)C(CCCCC)=C(NCc2ccccc2)C1=O] |
| 93 | CBD-Q (V) | 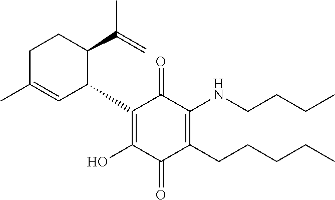 [SMILES: C=C(C)[C@@H]1CCC(C)=C[C@H]1C1=C(O)C(=O)C(CCCCC)=C(NCCCC)C1=O] |
| 94 | HU-410 | 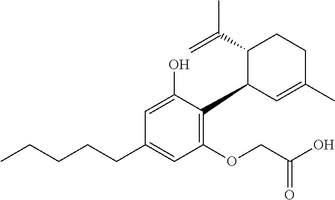 [SMILES: C=C(C)[C@@H]1CCC(C)=C[C@H]1c1c(O)cc(CCCCC)cc1OCC(=O)O] |

Sub-type: Other

| | | |
|---|---|---|
| 95 | Cannabicoumaronone (CBCON-C5) | 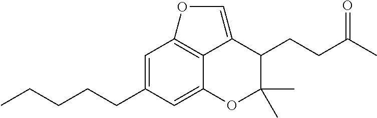 [SMILES: CCCCCc1cc2c3c(coc3c1)C(CCC(C)=O)C(C)(C)O2] |
| 96 | Cannabicitran (CBT-C5) | 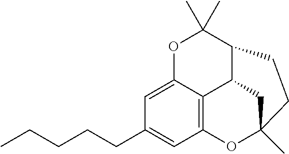 [SMILES: CCCCCc1cc2c3c(c1)O[C@@]1(C)CC[C@H]([C@@H]3C1)C(C)(C)O2] |
| 97 | RR-trans-Cannabitriol (RR-trans-CBT-C5) | 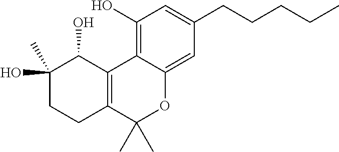 [SMILES: CCCCCc1cc(O)c2c(c1)OC(C)(C)C1=C2[C@@H](O)[C@](C)(O)CC1] |
| 98 | SS-trans-Cannabitriol (SS-trans-CBT-C5) | 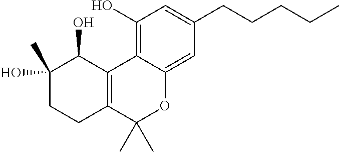 [SMILES: CCCCCc1cc(O)c2c(c1)OC(C)(C)C1=C2[C@H](O)[C@@](C)(O)CC1] |
| 99 | RS-cis-Cannabitriol (RS-cis-CBT-C5) | 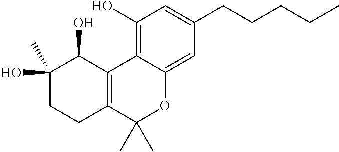 [SMILES: CCCCCc1cc(O)c2c(c1)OC(C)(C)C1=C2[C@H](O)[C@](C)(O)CC1] |
| 100 | SR-cis-Cannabitriol (SR-cis-CBT-C5) | 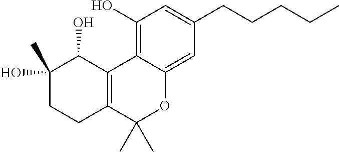 [SMILES: CCCCCc1cc(O)c2c(c1)OC(C)(C)C1=C2[C@@H](O)[C@@](C)(O)CC1] |
| 101 | RR-trans-Cannabitriol- C3 (RR-trans-CBT-C3) | 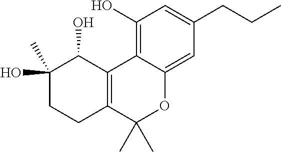 [SMILES: CCCc1cc(O)c2c(c1)OC(C)(C)C1=C2[C@@H](O)[C@](C)(O)CC1] |
| 102 | SS-trans-Cannabitriol- C3 (SS-trans-CBT-C3) | 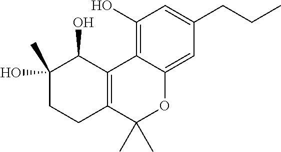 [SMILES: CCCc1cc(O)c2c(c1)OC(C)(C)C1=C2[C@H](O)[C@@](C)(O)CC1] |
| 103 | 8,9-Dihydroxy- Δ6a(10a)- tetrahydrocannabinol | 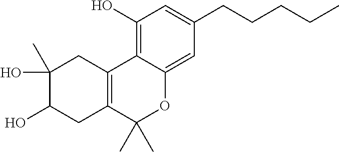 [SMILES: CCCCCc1cc(O)c2c(c1)OC(C)(C)C1=C2CC(C)(O)C(O)C1] |
| 104 | Cannabidiolic acid A cannabitriol ester (CBDA-C5 9-OH-CBT- C5-ester) | 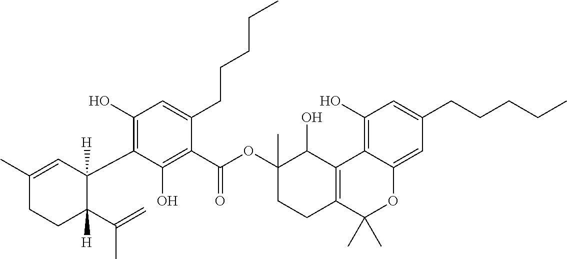 [SMILES: C=C(C)[C@@H]1CCC(C)=C[C@H]1c1c(O)cc(CCCCC)c(C(=O)OC2(C)CCC3=C(c4c(O)cc(CCCCC)cc4OC3(C)C)C2O)c1O] |
| 105 | (−)-6a,7,10a- Trihydroxy-Δ9- tetrahydrocannabinol ((−)-Cannabitetrol) | 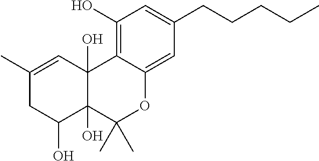 [SMILES: CCCCCc1cc(O)c2c(c1)OC(C)(C)C1(O)C(O)CC(C)=CC21O] |
| 106 | (−)-(6aR,9S,10S,10aR)- 9,10-Dihydroxy- hexahydrocannabinol, Cannabiripsol (Cannabiripsol-C5) | 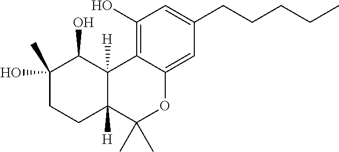 [SMILES: CCCCCc1cc(O)c2c(c1)OC(C)(C)[C@@H]1CC[C@](C)(O)[C@@H](O)[C@@H]21] |
| 107 | Cannabinol methyl ether (CBNM) | 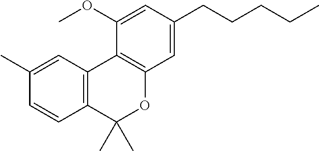 [SMILES: CCCCCc1cc(OC)c2c(c1)OC(C)(C)c1ccc(C)cc1-2] |
| 108 | 7-OH-CBD | 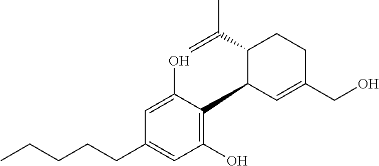 [SMILES: C=C(C)[C@@H]1CCC(CO)=C[C@H]1c1c(O)cc(CCCCC)cc1O] |
| 109 | 2′O-Glucuronide-CBD | 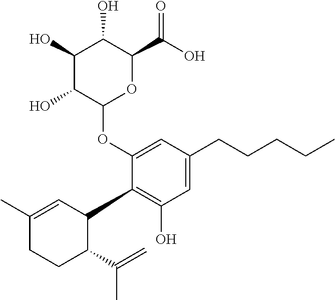 |
| 110 | Machaeridiol B | 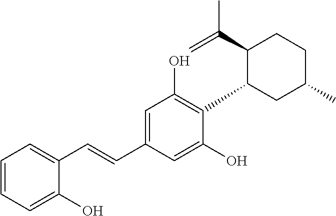 [SMILES: C=C(C)[C@H]1CC[C@H](C)C[C@@H]1c1c(O)cc(/C=C/c2ccccc2O)cc1O] |
| 111 | Ferrugiene C | 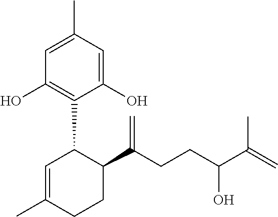 [SMILES: C=C(C)C(O)CCC(=C)[C@H]1CCC(C)=C[C@@H]1c1c(O)cc(C)cc1O] |
| 112 | Linderatin | 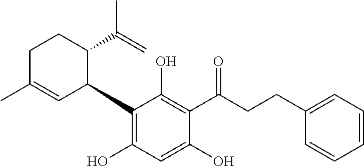 [SMILES: C=C(C)[C@H]1CCC(C)=C[C@@H]1c1c(O)cc(O)c(C(=O)CCc2ccccc2)c1O] |
| 113 | HU-465 | 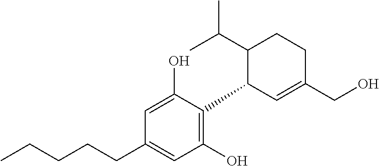 [SMILES: CCCCCc1cc(O)c([C@H]2C=C(CO)CCC2C(C)C)c(O)c1] |
| 114 | 7-OH-DMH-CBD | 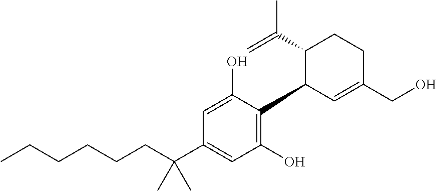 [SMILES: C=C(C)[C@@H]1CCC(CO)=C[C@H]1c1c(O)cc(C(C)(C)CCCCCC)cc1O] |
| 115 | DMH-CBDD | 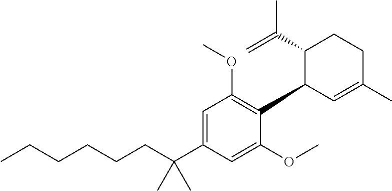 [SMILES: C=C(C)[C@@H]1CCC(C)=C[C@H]1c1c(OC)cc(C(C)(C)CCCCCC)cc1OC] |
| 116 | HUF-102 | 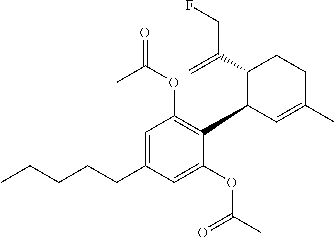 [SMILES: C=C(CF)[C@@H]1CCC(C)=C[C@H]1c1c(OC(C)=O)cc(CCCCC)cc1OC(C)=O] |
| 117 | CBD-diAcetate | 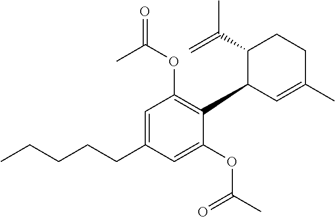 [SMILES: C=C(C)[C@@H]1CCC(C)=C[C@H]1c1c(OC(C)=O)cc(CCCCC)cc1OC(C)=O] |
| 118 | CBDD | 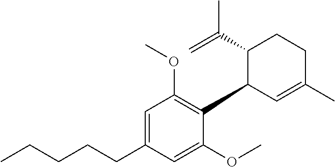 [SMILES: C=C(C)[C@@H]1CCC(C)=C[C@H]1c1c(OC)cc(CCCCC)cc1OC] |
| 119 | HU-444 | 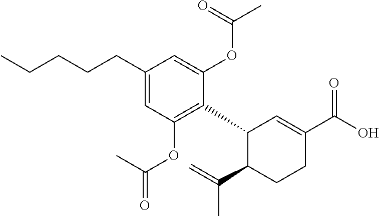 [SMILES: C=C(C)[C@@H]1CCC(C(=O)O)=C[C@H]1c1c(OC(C)=O)cc(CCCCC)cc1OC(C)=O] |
| 120 | HU-432 | 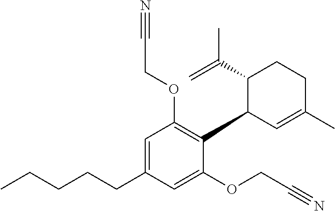 [SMILES: C=C(C)[C@@H]1CCC(C)=C[C@H]1c1c(OCC#N)cc(CCCCC)cc1OCC#N] |
| 121 | ALL00102 | 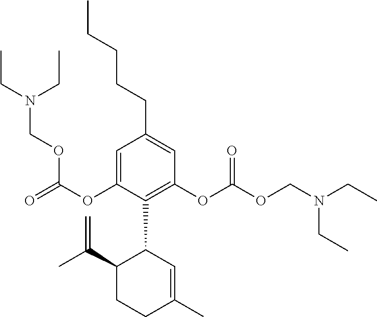 [SMILES: C=C(C)[C@@H]1CCC(C)=C[C@H]1c1c(OC(=O)OCN(CC)CC)cc(CCCCC)cc1OC(=O)OCN(CC)CC] |
| 122 | ALL00147 | 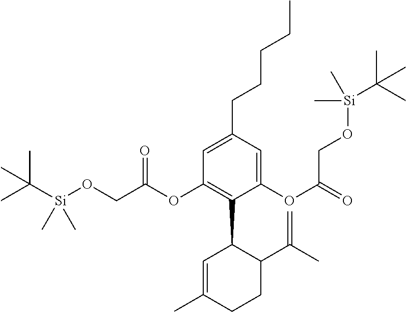 [SMILES: C=C(C)C1CCC(C)=C[C@H]1c1c(OC(=O)CO[Si](C)(C)C(C)(C)C)cc(CCCCC)cc1OC(=O)CO[Si](C)(C)C(C)(C)C] |
| 123 | CBD-aldehyde- diacetate | 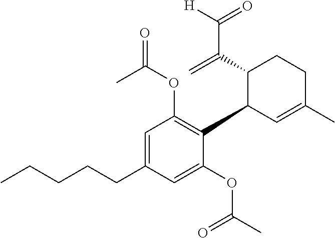 [SMILES: C=C(C=O)[C@@H]1CCC(C)=C[C@H]1c1c(OC(C)=O)cc(CCCCC)cc1OC(C)=O] |
| 124 | 6-Oxo-CBD-diacetate | 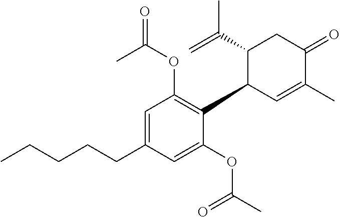 [SMILES: C=C(C)[C@@H]1CC(=O)C(C)=C[C@H]1c1c(OC(C)=O)cc(CCCCC)cc1OC(C)=O] |
| 125 | 6-Hydroxy-CBD- triacetate | 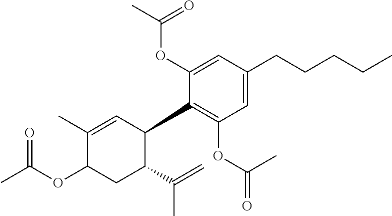 [SMILES: C=C(C)[C@@H]1CC(OC(C)=O)C(C)=C[C@H]1c1c(OC(C)=O)cc(CCCCC)cc1OC(C)=O] |
| 126 | 9-Hydroxy-CBD- triacetate | 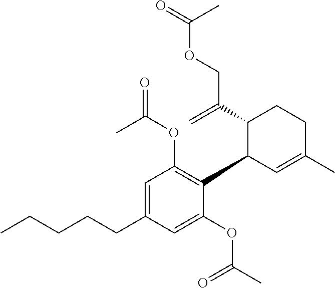 [SMILES: C=C(COC(C)=O)[C@@H]1CCC(C)=C[C@H]1c1c(OC(C)=O)cc(CCCCC)cc1OC(C)=O] |
| 127 | Cannabimovone | 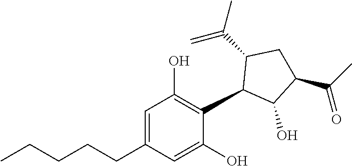 [SMILES: C=C(C)[C@@H]1C[C@@H](C(C)=O)[C@H](O)[C@H]1c1c(O)cc(CCCCC)cc1O] |

Provided herein are compositions comprising a polymer of one or more cannabinoid types. In some embodiments, the composition comprises a polymer of a single type of cannabinoid or derivative listed in TABLE 1. In some embodiments, the composition comprises a polymer of a plurality of types of cannabinoid or derivative listed in TABLE 1.

### Cannabinoid Polymers

As used herein the term “cannabinoid polymer(s)” and “polycannabinoid(s)” refer to a polymer comprising plurality of cannabinoid units. In some embodiments, a polycannabinoid polymer provided herein comprises a plurality of cannabinoid units, specifically phytocannabinoid units. In some embodiments, the cannabinoid polymer is a polymer comprising a plurality of cannabinoid units of the formula:

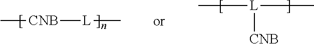

wherein:
CNB is a cannabinoid unit,
L is a linking group; and
n represents the number of repeat units wherein n is at least 2.

The cannabinoid units may be the same or different. In certain embodiments, each cannabinoid unit is independently CBG, CBD, CBC, CBND, DHCBD, CBG-R, CBD-R, CBC-R, CBND-R, DHCBD-R wherein the cannabinoid unit is bound to the linking group via hydroxyl groups, acid groups, or ester groups on the cannabinoid unit before polymerization. In some embodiments, a composition provided herein comprises a polymer comprising at least 50, 60, 70, 80, 90, 100 or more cannabinoid units. In some embodiments, a composition provided herein comprises a polymer comprising cannabinoid units polymerized from hemp oil. In some embodiments, a composition provided herein comprises a polymer comprising cannabinoid units polymerized from hemp oil, and also a polycannabinoid comprising less than 20 cannabinoid units, optionally 10 or less cannabinoid units, further optionally 6 or less cannabinoid units. Additional cannabinoids and cannabinoid derivatives can be found, for example, in Morales P, Reggio PH and Jagerovic N (2017) An Overview on Medicinal Chemistry of Synthetic and Natural Derivatives of Cannabidiol. Front. Pharmacol. 8:422, the contents of which are incorporated herein in their entirety by reference. Cannabinoid abbreviations are listed in TABLE 2.

TABLE 2
Cannabinoids and abbreviations.

| Cannabinoid | |
|---|---|
| Abbreviation | Cannabinoid |
| CBC | cannabichromene |
| CBC-R | substituted cannabichromene |
| CBD | cannabidiol |
| CBD-R | substituted cannabidiol |
| CBG | cannabigerol |
| CB G-R | substituted cannabigerol |
| DHCBD | dihydrocannabidiol |
| DHCBD-R | substituted dihydrocannabidiol |
| CBND | cannabinodiol |
| CBND-R | substituted cannabinodiol |

In some embodiments, each cannabinoid unit may be the same or different, each independently may be selected from those listed in TABLE 1, TABLE 2, or a derivative of any of these, and each has one of the following structures before polymerization, wherein the R group is C₁-C₁₀ alkyl optionally substituted with one or more heteroatoms, a heterocycloalkyl group, or a heteroaryl group, specifically C₁-C₆ alkyl, and more specifically n-pentyl or n-propyl; for the naturally occurring phytocannabinoids like CBD and CBG, R=methyl, ethyl, propyl, butyl, pentyl, hexyl, 4′-(3-carboxypropyl)-, 4′-(4-hydroxybutyl), 1,1-dimethylheptyl, 4′-[2-(1H-1,2,3-triazol-yl)ethyl]-, 4′-(2-morpholinoethyl)-, 4′-(2-ethoxyethyl)-:

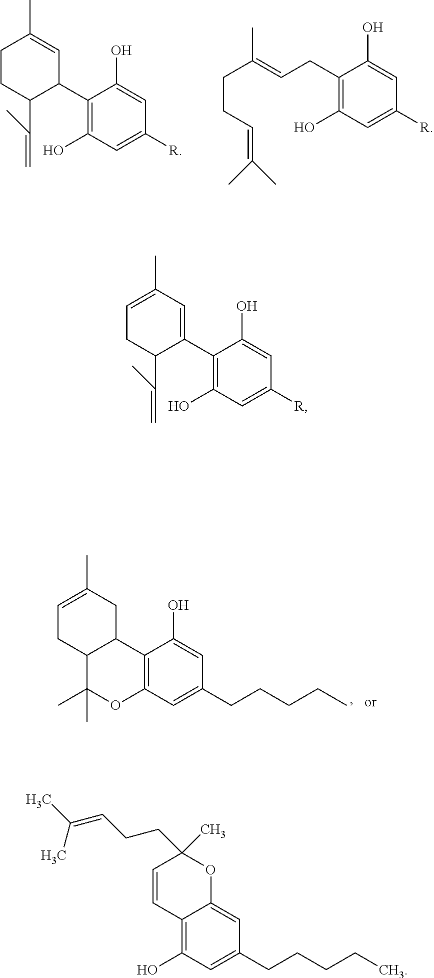

In some embodiments, each cannabinoid unit is the same and each has one of the structures listed in TABLE 1 before polymerization. In some embodiments, each cannabinoid unit is different and has one of the structures listed in TABLE 1 before polymerization. In some embodiments, a plurality of cannabinoid units of the structures listed in TABLE 1 before polymerization are incorporated into a composition. Polymers described herein may be formed by reacting the hydroxyl or other reactive functionalities, such as the diacetate or similar esters made from the hydroxyls on the cannabinoid unit or cannabinoid derivative with an electrophilic difunctional comonomer to produce the linkers, L. In some embodiments, the linking group which generally binds the cannabinoid unit are via linear or branched hydrocarbon chains containing from 3 to 50 carbon atoms, optionally interrupted with one or more oxygen atoms, these chains can be alkyl, alkenyl or alkynyl chains containing from 3 to 50 carbon atoms, or else polyether chains containing from 3 to 50 carbon atoms, it being possible for these chains to be substituted with hydrophilic groups (hydroxyl groups, for example). The chains binding the cannabinoid units to one another contain at least 3 carbon atoms and specifically from 4 to 50 carbon atoms, the shortest path between two cannabinoid units specifically consisting of a chain containing between 3 and 8 carbon atoms. Advantageously, the linking groups which link two cannabinoid units to one another may include linking groups of the general formula —O—(CH₂—CHOR¹—CH₂)ₘ—O—, where m is an integer between 1 and 50 (generally between 2 and 10) and where, in each of the n units (CH₂—CHOR¹—CH₂), R¹ denotes either a hydrogen atom or a —CH₂—CHOH—CH₂—O— chain bound to a cannabinoid unit of the polymer. In some embodiments, the polymers are be obtained by coupling of cannabinoid molecules with bifunctional compounds capable of forming covalent bonds with the hydroxyl groups of the cannabinoid. For example, they may be dicarboxylic acids such as citric acid, sebacic acid, fumaric acid, glutamic acid, maleic acid, malic acid, malonic acid, oxalic acid, succinic acid, glutaric acid, terephthalic acid, isophthalic acid, oxaloacetic acid, phthalic acid, adipic acid or butanedioic acid. Representative specific examples of the linking groups are those monomers which polymerize to form vinyl polymers, polyurethanes, polyesters, polyethers, polyamides, polyimides, polyamino acids, polypeptides, polysaccharides, and the like. When the linking group is a vinyl monomer, specific examples of the vinyl polymer include (meth)acrylic monomers, styrene monomers, (meth)acrylamide monomers, ethylene monomers, propylene monomers, oxyethylene monomers, ethylene glycol monomers, propylene glycol monomers, monomers of vinyl alcohol, vinyl acetate monomers, vinyl chloride monomers, and the like. As used herein, (meth)acrylate refers to acrylate or methacrylate, and (meth)acrylic refers to methacrylic or acrylic.

Examples of (meth)acrylic monomers include (meth)acrylic acids and salts thereof, and (meth)acrylic acid esters such as methyl (meth)acrylate, ethyl (meth)acrylate, hydroxymethyl (meth)acrylate, and hydroxyethyl (meth)acrylate. Examples of styrene monomers include styrene, styrene sulfonates, and the like. Examples of (meth)acrylamide polymers include (meth)acrylamides, and (meth)acrylamide derivatives such as dimethyl (meth)acrylamide, diethyl (meth)acrylamide, N-isopropylacrylamide, and N-benzylacrylamide. The linking group monomers are not limited to those mentioned above as examples. Conventionally known vinyl monomers are also usable.

The cannabinoid polymer may be a homopolymer, or a copolymer obtained by copolymerizing monomers. When the cannabinoid polymer is a copolymer with one or more additional polymers, the additional polymers may be any of random copolymers, alternating copolymers, graft copolymers, or block copolymers. The side chain of the additional polymers may be substituted with a functional group. That is, as long the desired effect of the cannabinoid polymer is not impaired, the main chain and side chains of the additional polymers may be modified with other substituents by chemical bonds or the like.

In some embodiments, the cannabinoid monomer is incorporated into a thermoplastic polymer or a biodegradable polymer. Suitable thermoplastic polymers include, but are not limited to polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid) polymers, polymaleic anhydrides, poly(methylvinyl) ethers, poly(amino acids), chitin, chitosan, polythiocarbonates, polythiourethanes, and copolymers, terpolymers, or combinations or mixtures of the above materials.

Examples of biodegradable polymers and oligomers suitable for use in the compositions and methods described herein include, but are not limited to: poly(lactide)s; poly(glycolide)s; poly(lactide-co-glycolide)s; poly(lactic acid)s; poly(glycolic acid)s; and poly(lactic acid-co-glycolic acid)s; poly(caprolactone)s; poly(malic acid)s; polyamides; polyanhydrides; polyamino acids; polyorthoesters; polyetheresters; polycyanoacrylates; polyphosphazines; polyphosphoesters; polyesteramides; polydioxanones; polyacetals; polyketals; polycarbonates; polyorthocarbonates; degradable polyurethanes; polyhydroxybutyrates; polyhydroxyvalerates; polyalkylene oxalates; polyalkylene succinates; chitins; chitosans; oxidized celluloses; and copolymers, terpolymers, blends, combinations or mixtures of any of the above materials.

As used herein, “hydrophobic” refers to a polymer that is substantially not soluble in water. As used herein, “hydrophilic” refers to a polymer that may be water-soluble or to a polymer having affinity for absorbing water, but typically not when covalently linked to the hydrophobic component as a co-polymer, and which attracts water into the device.

The cannabinoid unit can be incorporated into hydrophilic polymers. Hydrophilic polymers suitable for use herein can be obtained from various commercial, natural or synthetic sources well known in the art. Suitable hydrophilic polymers include, but are not limited to: polyanions including anionic polysaccharides such as alginate; agarose; heparin; polyacrylic acid salts; polymethacrylic acid salts; ethylene maleic anhydride copolymer (half ester); carboxymethyl amylose; carboxymethyl cellulose; carboxymethyl dextran; carboxymethyl starch; carboxymethyl chitin/chitosan; carboxy cellulose; 2,3-dicarboxycellulose; tricarboxycellulose; carboxy gum arabic; carboxy carrageenan; carboxy pectin; carboxy tragacanth gum; carboxy xanthan gum; carboxy guar gum; carboxy starch; pentosan polysulfate; curdlan; inositol hexasulfate; beta.-cyclodextrin sulfate; hyaluronic acid; chondroitin-6-sulfate; dermatan sulfate; dextran sulfate; heparin sulfate; carrageenan; polygalacturonate; polyphosphate; polyaldehydo-carbonic acid; poly-1-hydroxy-1-sulfonate-propen-2; copolystyrene maleic acid; mesoglycan; sulfopropylated polyvinyl alcohols; cellulose sulfate; protamine sulfate; phospho guar gum; polyglutamic acid; polyaspartic acid; polyamino acids; and any derivatives or combinations thereof. One skilled in the art will appreciate other hydrophilic polymers that are also within the scope of the present disclosure.

The cannabinoid unit can be incorporated into various water-soluble polymers. Water-soluble polymers include, but are not limited to: poly (alkyleneglycol), polyethylene glycol (“PEG”); propylene glycol; ethylene glycol/propylene glycol copolymers; carboxylmethylcellulose; dextran; polyvinyl alcohol (“PVOH”); polyvinyl pyrrolidone; poly (alkyleneamine)s; poly (alkyleneoxide)s; poly-1,3-dioxolane; poly-1,3,6-trioxane; ethylene/maleic anhydride copolymers; polyaminoacids; poly (n-vinyl pyrrolidone); polypropylene oxide/ethylene oxide copolymers; polyoxyethylated polyols; polyvinyl alcohol succinate; glycerin; ethylene oxides; propylene oxides; poloxamers; alkoxylated copolymers; water soluble polyanions; and any derivatives or combinations thereof. In addition, the water-soluble polymer may be of any suitable molecular weight, and may be branched or unbranched.

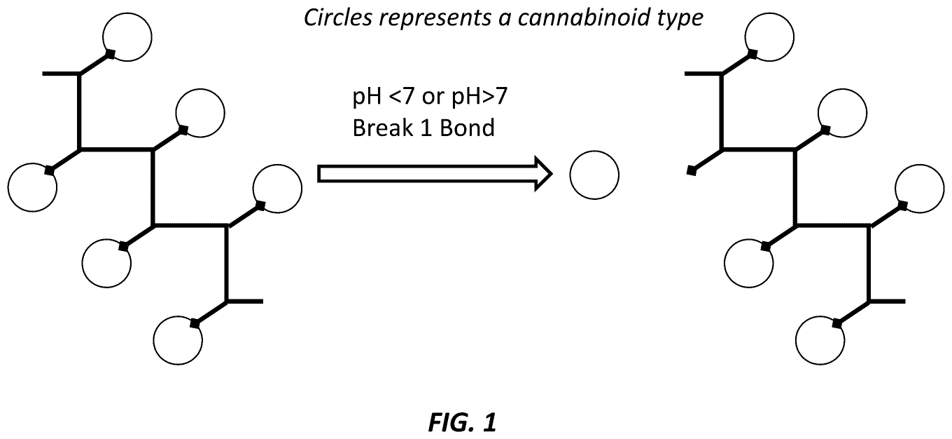

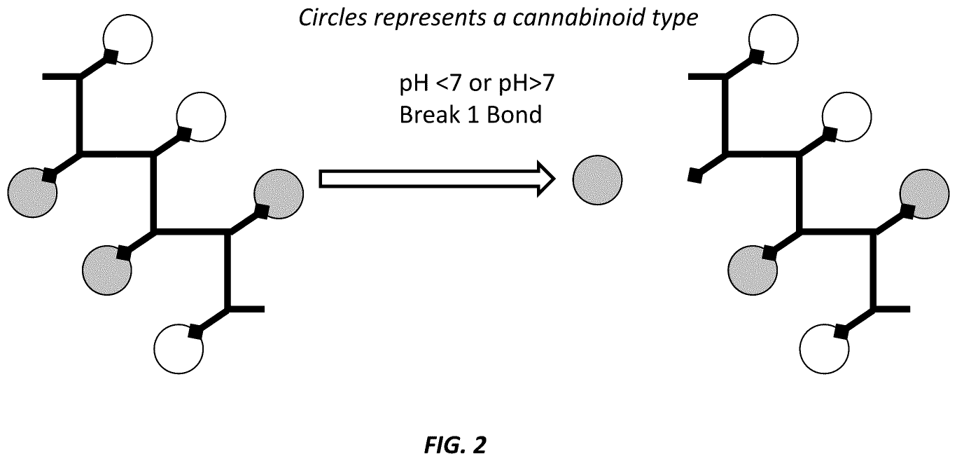

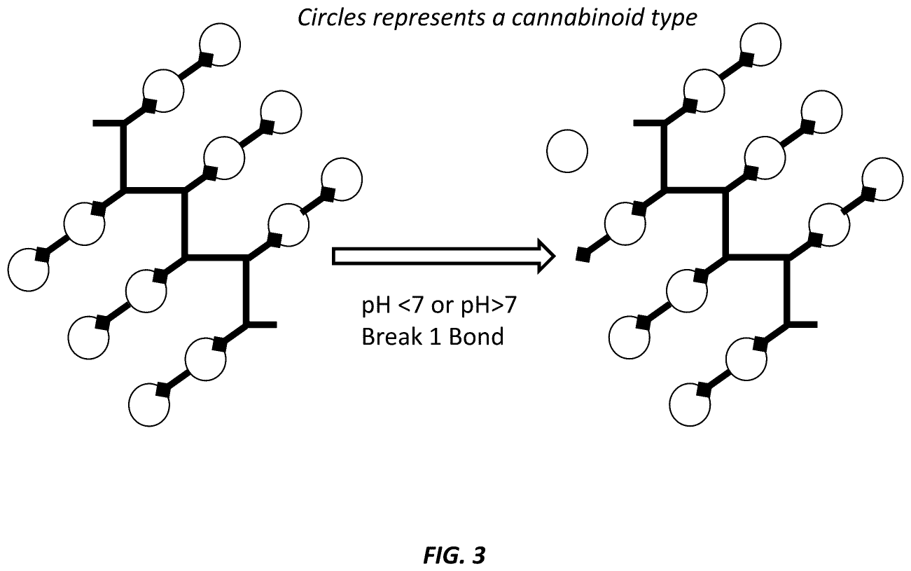

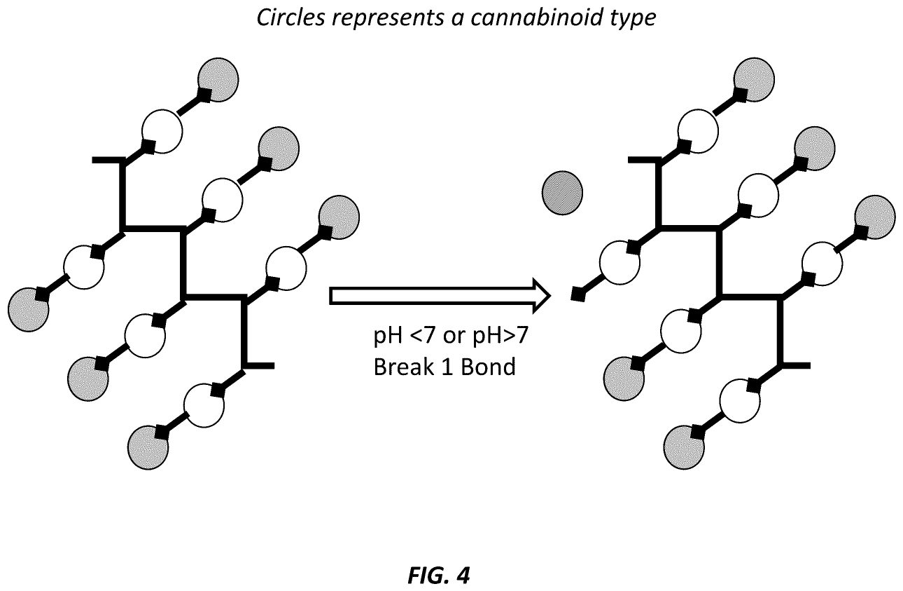

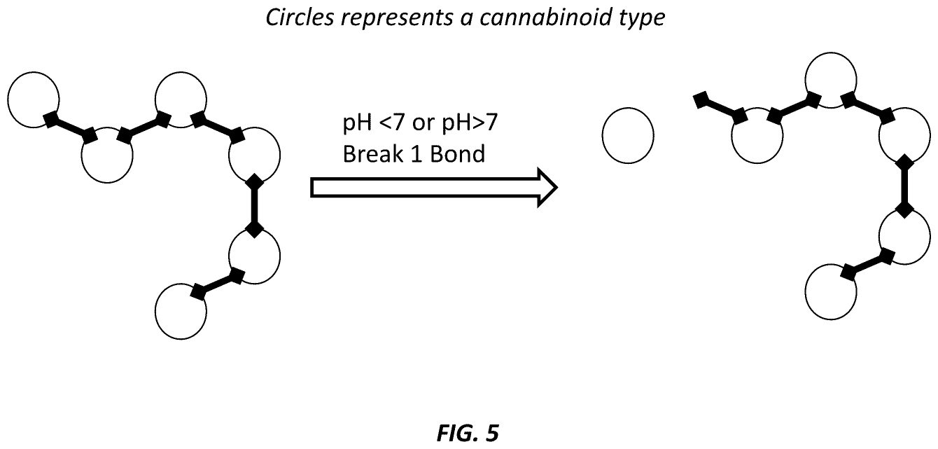

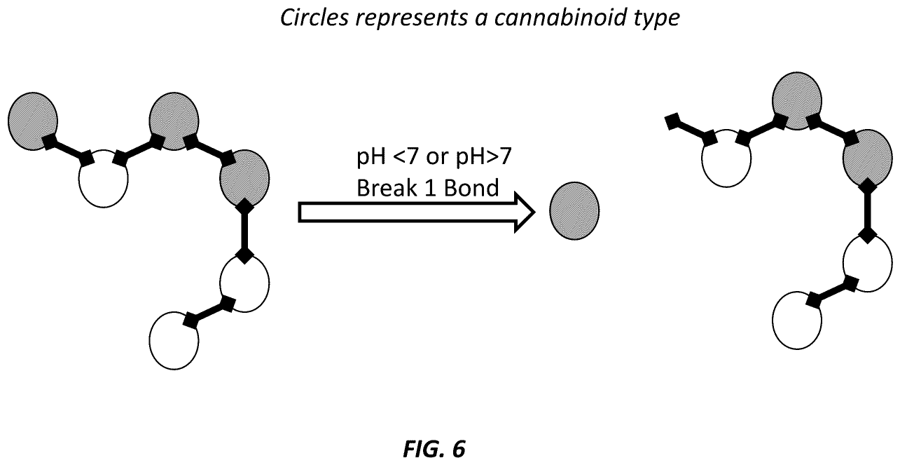

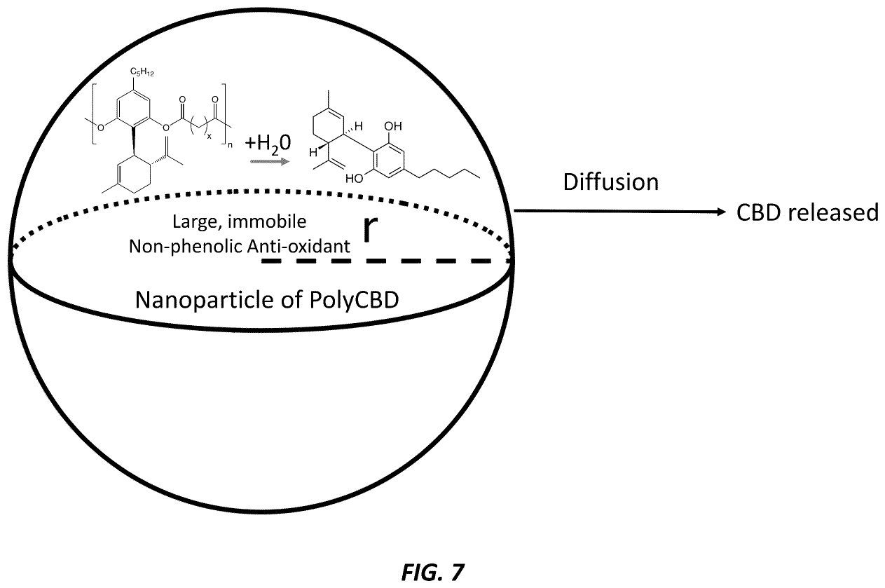

Referring to FIG. 1, it is a schematic representation is provided of a branched cannabinoid polymer under acidic or basic conditions resulting in the release of a singular cannabinoid molecule. The probability of releasing a certain cannabinoid type is 100%. Referring to FIG. 2, it is a schematic representation of a branched cannabinoid polymer under acidic or basic conditions resulting in the release of a singular cannabinoid molecule (or other previously covalently bound API), where more than one species of cannabinoid is represented. The probability of releasing a certain cannabinoid type is 50% in this arrangement. Referring to FIG. 3, it is a schematic representation of an unbranched cannabinoid polymer under acidic or basic conditions resulting in the release of a singular cannabinoid molecule. The probability of releasing the cannabinoid is 50% as increasing daisy chain length to scaffold decreases probability of release. Referring to FIG. 4, it is a schematic representation of an unbranched cannabinoid polymer under acidic or basic conditions resulting in the release of a singular cannabinoid molecule (or other previously covalently bound API), where more than one species of cannabinoid is represented. The probability of releasing a certain cannabinoid type is 50% in this arrangement. Referring to FIG. 5, it is a schematic representation of a daisy-chain type cannabinoid polymer under acidic or basic conditions resulting in the release of a singular cannabinoid molecule. The probability of releasing an active cannabinoid 20% for daisy chain of 6 drug molecules. Referring to FIG. 6, it is a schematic representation of a daisy-chain type cannabinoid polymer under acidic or basic conditions resulting in the release of a singular cannabinoid molecule (or other previously covalently bound API), where more than one species of cannabinoid is represented. The probability of releasing the shaded cannabinoid is 10% for daisy chain of 6 drug molecules (3 shaded and 3 non-shaded). By increasing the ratio of shaded:non-shaded, then increase releasing the shaded cannabinoid probability. Referring to FIG. 7, it is a schematic representation of a polyCBD nanoparticle and the release of a CBD monomer with the addition of water (H₂O) in conjunction with a diffusion process. Diffusion is fastest for smaller molecules, such as CBD upon hydrolysis, a CBD dimer (n=2, x=8 for example) releases a CBD molecule. In another exemplary arrangement, a nanoparticle made of high molecular weight PolyCBD (n=>20) is loaded with bioactive PolyCBD (n=2 to 6), optionally with non-polymerized CBD. Implications for such a profile include, without limitation, tunability of functional aspects. For example, polyCBD is not a phenolic anti-oxidant, but when CBD is released, it is a phenolic anti-oxidant. Such tunability has implications for chemotherapy treatment, amongst other applications.

In some embodiments, the cannabinoid polymers are endcapped with a suitable monomer having a singularly reactive monomer. The endcap can be any group which does not reduce efficacy of the cannabinoid units compared to an equivalent polymer without the endcap. In some embodiments, the endcap groups can be, independently, a linear or branched alcohol, or a singly reactive cannabinoid unit, for example, a cannabinoid unit having only one hydroxy group, one acid group, or one ester group. In some embodiments, the endcap may have additional reactive cites which are protected during the reaction with the polymer and are later deprotected to provide additional reactive functionality to the polymer. In some embodiments, each singly reactive cannabinoid unit has the structure:

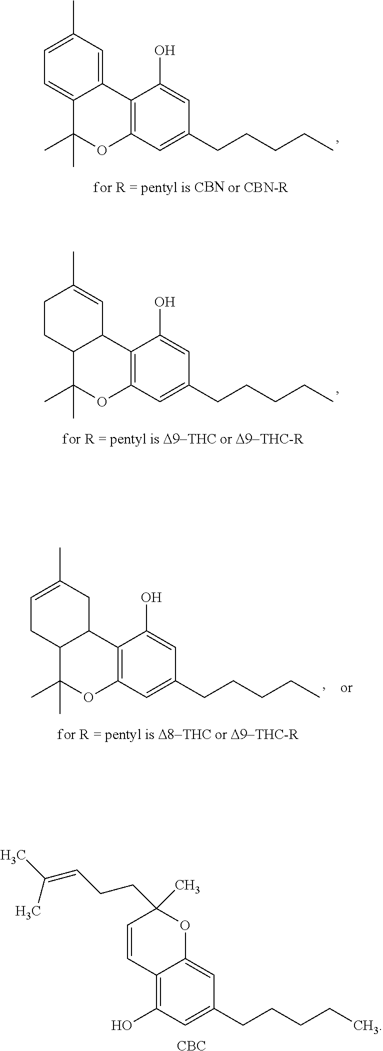

In some embodiments, the cannabinoid has a high affinity for an endocannabinoid receptor. In some embodiments, the cannabinoid has a high affinity for CB1, CB2, or a combination thereof.

In some embodiments, the cannabinoid is a synthesized cannabinoid, not found in nature. In some embodiments the cannabinoid is an analogue of phytocannabinoid. In some embodiments, the cannabinoid comprises an isothiocyanate functionality.

In some embodiments, the cannabinoid has photoactivatable groups covalently bound to the cannabinoid ring core.

In some embodiments, the cannabinoid is commercially available.

In some embodiments, the cannabinoid is radiolabeled.

In some embodiments, the cannabinoid is a tricyclic cannabinoid.

In some embodiments the cannabinoid has lipophilic properties.

In some embodiments, the cannabinoid is an adamantyl cannabinoid.

In some embodiments the cannabinoid has polar properties.

In some embodiments, the cannabinoid is an oxa-adamantyl cannabinoid.

In some embodiments, cannabinoid polymers described herein have a number average molecular weight of about 1,000 daltons to about 60,000 daltons. In some embodiment, the cannabinoid polymers have a number average molecular weight of about 5,000 daltons to about 55,000 daltons, a number average molecular weight of about 6,000 daltons to about 50,000 daltons, a number average molecular weight of about 7,000 daltons to about 50,000 daltons, a number average molecular weight of about 9,000 to about 40,000 daltons, or a number average molecular weight of about 10,000 to about 30,000 daltons. In some embodiments, cannabinoid polymer sizes are calculated using a mass of approximately 320 Daltons for a cannabinoid and the range of molecular weights o the aliphatic diacids going from oxalic acid to dodecanoic acid.

In some embodiments the cannabinoid polymer is a polyester polymer.

In some embodiments, bioactivity of polycannabinoids is evaluated. In some embodiments, the polycannabinoid is not cytotoxic. In some embodiments, the release of a cannabinoid from a polycannabinoid polymer results in the presence of an antioxidant. In some embodiments, the release of a cannabinoid from a polycannabinoid polymer results in the presence of an antioxidant, and an anti-inflammatory effect when administered to a subject.

In some embodiments, the polymer comprising a plurality of cannabinoid units has daisy-chain type connectivity, such as illustrated in FIGS. 3-6. For daisy chain type cannabinoid polymers, bioactive versions can be linked to a thermally or photopolymerizable functionality, described herein as a “linker”. In some embodiments, the linker is an acrylate molecule. In some embodiments, the acrylate is selected from at least one of: methylacrylate, methylmethacrylate, and 2-hydroxyethylmethacrylate. In some embodiments, under acidic or basic conditions, the polymer releases a cannabinoid monomer as depicted.

### Methods of Preparation of Cannabinoid Polymers

The following general preparative methods are presented to aid the reader in synthesizing the compounds, with more detailed particular examples being presented below in the experimental section describing the working examples. Where detail is not provided herein, the described cannabinoid polymers can be made according to conventional chemical methods, and/or as disclosed below, from starting materials which are either commercially available or producible according to routine, conventional chemical methods. General methods for the preparation of the compounds are given below, and the preparation of representative compounds is specifically illustrated in examples. The general methods to make cannabinoid polymers described herein are illustrated in Reaction Schemes 1-4.

The cannabinoid polymers may be formed by solventless procedures (melt polymerizations) as well as those requiring solvent including combinations of pure monomers if both are liquids (includes the melting of CBD or other cannabinoid to form a liquid, alternatively, the polymerization can be carried out in a solvent) or by interfacial polymerization.

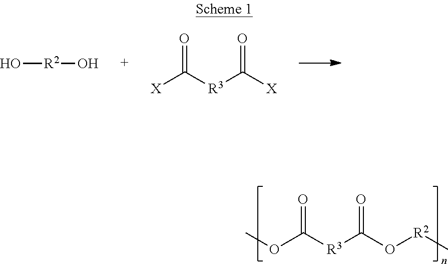

Scheme 1 presents a generic reaction scheme for the reaction of a cannabinoid diol monomer (HO—R²—OH) with a dicarbonyl monomer to produce a cannabinoid polyester. Equal equivalents of each will produce a high molecular weight polymer (Mn>20 kDa). The non-diol monomer could be a dicarboxylic acid, a diester, a dianhydride, a diacid chloride where X would be equal to —OH, O—R⁴, O—(C═O)—OR⁴ wherein R⁴ can be aliphatic, Cl, respectively. R³ could be aliphatic, branched aliphatic, halogenated (halogen includes fluorine, chlorine, bromine) aliphatic, halogenated branched aliphatic, aromatic, ethyleneoxy (linear or branched ether) or combinations thereof.

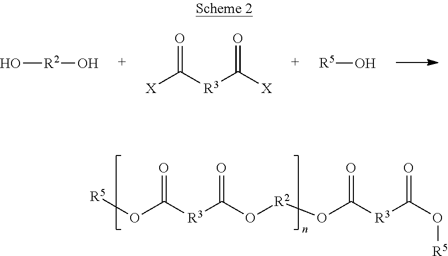

Scheme 2 presents a generic reaction scheme for the reaction of a cannabinoid diol monomer (HO—R²—OH) with a dicarbonyl monomer in the presence of a cannabinoid with single hydroxy (R⁵—OH) to produce a cannabinoid polyester with cannabinoid endcaps. Endcapping can control the molecule weight of the polymer and can control the ratio of the two cannabinoids. The non-diol monomer could be a dicarboxylic acid, a diester, a dianhydride, a diacid chloride where X would be equal to —OH, O—R⁴, O—(C═O)—OR⁴, Cl, respectively.

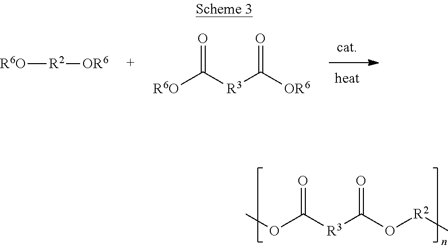

Scheme 3 shows a reaction in which diols are easily converted to (R⁶O—R²—OR⁶) a short ester such as a methyl or ethyl ester (R⁶=lower alkyl). The diester monomer can then be transesterified to produce a polyester.

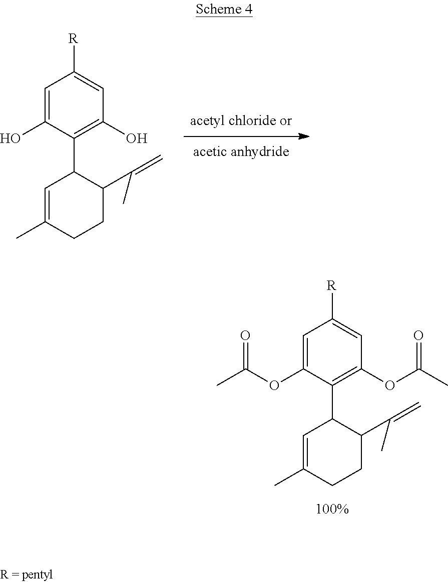

Scheme 4 shows, as a model for polymerization, CBD can be converted quantitatively to diacetyl CBD in accordance with the following reaction. Diacetyl CBD is a colorless liquid whereas CBD is a solid. Hence, diacetyl CBD can allow for a liquid phase polymerization without solvent with another monomer to produce a high molecular weight polymer. The other diols can undergo similar chemistry to make diacetyl monomers for transesterification.

The polycannabinoids can be altered by the type of polymer (polyester, polyurethane, polycarbonate) which will then alter the polymer properties. Flexibility in the backbone will result in low glass transition temperature (Tg) materials that will be rubbery at room temperature whereas reducing the flexibility will increase the Tg making them a glassy solid. Cannabinoids have an exact stereochemistry, so polymerization with a symmetrical comonomer can produce semi-crystalline polymers with the ability to be melt cast into films and fibers. Melt polymerization is also possible if the polymer generated is semicrystalline.

The cannabinoid polymers are thermally stable and stable against conversion of the target cannabinoid to another cannabinoid compound. Such thermostability provides advantageous features for anti-tampering of compositions having cannabinoid polymers. I

### Compositions and Carrier Systems

In some embodiments, provided herein are compositions comprising a polycannabinoid and a lipid or lipid derivative. In some embodiments the lipid is an oil. In some embodiments, provided herein are compositions comprising a polycannabinoid and an oil extract from a plant. In some embodiments, the oil is a vegetable oil. In some embodiments the oil is selected from at least one of: palm oil, soybean oil, rapeseed oil, sunflower seed oil, palm kernel oil, peanut oil, cottonseed oil, coconut oil, or olive oil. In some embodiments, the composition is a nanoformulation. In some embodiments, the nanoformulation may be used for the prevention of a disease. In some embodiments, the nanoformulation may be used for the management of a disease.

In some embodiments, compositions described herein further comprise a secondary bioactive compound. In some embodiments, the bioactive compound is a bioactive compound naturally occurring in vegetable oils. In some embodiments the bioactive compound naturally occurring in vegetable oils has nutraceutical properties. In some embodiments, the bioactive compound naturally occurring in vegetable oils is a lecithin phosphoglyceride. In some embodiments, the bioactive compound naturally occurring in vegetable oils is a carotenoid. In some embodiments, the bioactive compound naturally occurring in vegetable oils is a saturated fatty acid. In some embodiments, the bioactive compound naturally occurring in vegetable oils is a phytosterol. In some embodiments, the bioactive compound naturally occurring in vegetable oils is a tocopherol. In some embodiments, the bioactive compound naturally occurring in vegetable oils is a triacylglycerol (TAG).

In some embodiments, the cannabinoid polymer is in the form of a nanoparticle. In some embodiments, the nanoparticle is a CBD polymer (PolyCBD). In some embodiments, the nanoparticle is made of high molecular weight PolyCBD (comprising equal or greater than 20 cannabinoid units). In some embodiments, smaller cannabinoid polymers comprising about 2 to about 6 cannabinoid units are loaded into the nanoparticle. In some embodiments, smaller cannabinoid polymers comprising about 2 to about 10 cannabinoid units are loaded into the nanoparticle. In some embodiments the smaller cannabinoid polymers further comprise CBD. In some embodiments, the nanoparticle of polyCBD remains immobile. In some embodiments the nanoparticle of polyCBD is subject to diffusion. In some embodiments, the nanoparticle of polyCBD is exposed to water as shown in FIG. 7. In some embodiments, the nanoparticle of polyCBD comprising CBD or CBD dimers releases a free CBD molecule.

Without wishing to be bound by theory, cannabinoid polymers are non-phenolic anti-oxidants. Anti-oxidant activity of cannabinoid polymers can be attributed to the presence of a tertiary benzylic position. When a singular CBD molecule is released from the polymer, the singular CBD molecule is also an antioxidant. The antioxidant activity of singular CBD molecules are characterized as phenolic antioxidants. In some embodiments, nanoparticles of polyCBD are characterized as non-phenolic biopolymer anti-oxidant nanoparticles. In some embodiments, the nanoparticles limit oxidative stress. In some embodiments, the nanoparticles act as scavengers for reactive oxygen species (ROS) and reactive nitrogen species in subjects undergoing chemotherapy or other treatments resulting in oxidative stress processes in the body of the subject.

In some embodiments, the polyCBD nanoparticle is a biocompatible material. In some embodiments, a polyCBD nanoparticle described herein comprises a carrier matrix. In some embodiments, the carrier matrix is a hydrogel. In some embodiments, the carrier matrix provides a pH buffering capacity.

In some embodiments, fibers are provided comprising a cannabinoid polymer described herein. In some embodiments, films are provided comprising a cannabinoid polymer described herein. In some embodiments, meshes are provided comprising a cannabinoid polymer described herein. In some embodiments, transdermal patches are provided comprising a cannabinoid polymer described herein. In some embodiments, bandages are provided comprising a cannabinoid polymer described herein.

In some embodiments, the nanoparticle is selected from one of a: nanoliposome, nanoliposphere, nanoemulsion, solid lipid nanoparticle (SLN), nanostructured lipid carrier (NLC), polymeric nanoparticle, a hybrid nanoparticle, or an inorganic particle.

In some embodiments, a device comprises a composition described herein. Exemplary devices include surgical tools, containers, wraps, and filters. Exemplary surgical tools include syringes, staples, tapes, wires, strings, sutures, meshes, and tubes. Exemplary containers include bottles, vials, and capsules. Exemplary wraps include bandages and seals. Exemplary filters include membranes. In some embodiments, a composition is provided for comprising one or more polycannabinoids described herein, wherein the composition is in the form of a dissolvable tab, capsule, tablet, chewable, powders, liquid, semi solid, or solid. In some embodiments, the composition is in the form of an ointment.

### Pharmaceutical and Nutraceutical Compositions

In some embodiments, compositions described herein are incorporated into a pharmaceutical composition. In some embodiments, the pharmaceutical composition comprises one or more compositions described herein and a pharmaceutically acceptable excipient or pharmaceutically acceptable carrier. Pharmaceutically acceptable excipients include pharmaceutically acceptable salts.

The present disclosure includes, where applicable, the compositions comprising pharmaceutically acceptable salts. The term “pharmaceutically acceptable salt” is used throughout the specification to describe, where applicable, a salt form of one or more of the cannabinoid polymers described herein which are presented to increase the solubility of the compound in the gastric juices of the patient's gastrointestinal tract to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure. Exemplary pharmaceutically acceptable salts include acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

The composition as described herein may, in accordance with the disclosure, be administered in single or divided doses. The composition as described herein may, in accordance with the disclosure, be administered once or multiple times. Such administrations may be by oral, intravenous, intramuscular, subdermal, intradermal, subcutaneous, rectal, intranasal, parenteral or topical routes. The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Administration of the composition may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the composition from an oral route of administration. The most effective dosage form will depend upon the pharmacokinetics of the particular agent chosen as well as the severity of disease in the patient. Administration of composition according to the present disclosure as sprays, mists, or aerosols for intra-nasal, intra-tracheal or pulmonary administration may also be used. The present disclosure therefore also is directed to compositions comprising an effective amount of compounds as described herein, optionally in combination with a pharmaceutically or cosmetically acceptable carrier, additive or excipient. Compositions according to the present disclosure may be administered in immediate release, intermediate release or sustained or controlled release forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, exemplary carriers are physiological saline or phosphate buffered saline (PBS).

The compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

Alternatively, the compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax and water. In certain aspects of the disclosure, the compounds may be coated onto an implantable medical device, e.g., a stent which is to be surgically implanted into a patient in order to inhibit or reduce the likelihood of occlusion occurring in the stent in the patient. Other embodiments include a catheter or a catheter coating. Other exemplary implantable devices that can be coated with the polycannabinoids include orthopedic implants, interocular lenses, and the like where the antioxidant and antimicrobial properties of the cannabinoid units provide added benefit.

Alternatively, the compositions can be formulated in a suitable lotion or cream containing the active components suspended or dissolved in one or more pharmaceutically or cosmetically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

It may be desirable or necessary to introduce the composition to the patient via a mechanical delivery device. The construction and use of mechanical delivery devices for the delivery of such agents is well known in the art. Direct techniques for, for example, administering a drug directly to the brain usually involve placement of a drug delivery catheter into the patient's ventricular system to bypass the blood-brain barrier.

For ophthalmic use, the compositions may be formulated as micronized suspensions in isotonic, pH adjusted sterile saline, or, as solutions in isotonic, pH adjusted sterile saline, either with or without a preservative such as benzylalkonium chloride. Alternatively, for ophthalmic uses, the compositions may be formulated in an ointment such as petrolatum.

The compositions as described herein may also be administered by nasal aerosol or inhalation. Such compositions are prepared according to techniques well-known in the art of compound formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a composition as described herein that may be combined with the carrier materials to produce a single dosage form will vary depending upon the cannabinoid, the host and the disease treated, the particular mode of administration. The compositions can be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure. A particular advantage of the cannabinoid polymers described herein is the ability to prepare and administer accurate concentrations of a cannabinoid due to the polymer's stability against thermal degradation and stability against unwanted conversion of the target cannabinoid to other cannabinoid compounds.

It should also be understood that a specific dosage and treatment regimen for any particular patient will depend upon a variety of factors, including the activity of the specific compound employed, the age, body weight, general health, sex, diet, time of administration, rate of excretion, drug combination, and the severity of the particular disease or condition being treated.

In another aspect, a film comprises the cannabinoid polymer(s) described herein. In another aspect, a nonwoven fabric comprises fibers comprising the cannabinoid polymer(s). In certain embodiments, the films or nonwoven fabrics comprising the cannabinoid polymers described herein can be used in the fabrication of patches for transdermal delivery, bandages, wound dressing, sutures or for other topical or sublingual applications. In certain embodiments, the films or fibers are formed by melt processing of the cannabinoid polymers. Transdermal patches and wound dressings may be used to provide continuous or discontinuous infusion of the polymers and compounds in controlled amounts. Such patches may be constructed for continuous, pulsatile, or on demand delivery of such agents. In one aspect, a patch is made from the cannabinoid polymer(s). Transdermal patches and wound dressings may be a film or a sheet formed using the polymer. In certain embodiments, the film or the sheet of the polymer is further physically or chemically crosslinked or additional layers or sheets including, but not limited to, adhesive layers, protective layers, release layers, backing layers, and the like. The transdermal patches and wound dressings, particularly in film-form or sheet-form, may or may not be porous. In some embodiments, the thickness of the film-form or sheet-form transdermal patches or wound dressings is at least about 10 μm, from about 75 μm to about 5,000 μm, from about 100 μm to about 2,000 μm, or from about 200 μm to 1,000 μm. In some embodiments, the thickness of the film-form or sheet-form transdermal patches or wound dressings is at least 10 μm, from 75 μm to 5,000 μm, from 100 μm to 2,000 μm, or from 200 μm to 1,000 μm. The transdermal patches and wound dressings can be formed into a sheet form by extrusion molding. Any conventional methods such as inflation molding, T-die molding, lamination molding, etc., can be used as the extrusion molding, and since a high-temperature extrusion and quick cooling are applicable, the film or sheet of the polymer having an excellent optical property can be obtained. Furthermore, from the standpoints that the production speed can be increased due to the good cooling efficiency and the thickness of the sheet formed can be easily controlled, T-die molding can be employed. In addition, a single screw extruder or a twin screw extruder can be used as the extruder. By properly controlling the molding conditions such as the molding temperature, the die lip width, the extrusion speed, the drawing speed, etc., the thickness of the film or sheet can be controlled. Any conventional methods such as a calendar method, a casting method, an extrusion method, etc., can be used as the film-forming method of the film or the sheet of the polymer described herein. The film or the sheet-thus obtained is cut into a desired form and can be used as a patch or wound dressing. A mesh or porous film or sheet can be obtained by punching the film or sheet with a mold or by molding in a casting method with a mesh-form mold. Also, a porous dressing can be obtained by swelling the film or the sheet by absorbing water and then dry freezing the swelled film or sheet. A mesh or porous sheet has a gas permeability factor which can be controlled for the particular application. In certain embodiments, the patch or wound dressing may have a backing material or backing layer to improve the strength, the fixing property, the form-retention property, the adhesive property, etc. In this case, the size of the backing material is same as or larger than the size of each dressing of each form. Any conventional materials can be used as the material of the backing material. Specific examples of the material for the backing material are papers, nonwoven fabrics, cotton fabrics, synthetic resin fabrics, synthetic resin films, synthetic resin foams, mesh-form or network papers, nonwoven fabrics, cotton fabrics, synthetic resin fabrics, or synthetic resin films, and the surgical tapes, the medical pressure-sensitive adhesive sheets, pressure-sensitive adhesive dressings, etc., using the above films or sheets as a substrate. Of those, the materials having excellent gas permeability and moisture permeability are more preferred. Similarly, in certain embodiments, the patch or wound dressing may have an adhesive material or layer, with or without a release layer. Any conventional materials can be used as the material of the adhesive layer.

In some embodiments, a composition comprising one or more polycannabinoids described herein further comprises at least one terpene. In some embodiments, the composition comprises one or more of the following terpenes: D-Limonene, geraniol, humulene, linalool, myrcene, terpineol, terpinolene, α-Pinene, or β-Caryophyllene. In some embodiments, the composition comprises one or more polycannabinoids comprising a cannabinoid listed in TABLE 1 or TABLE 2 prior to polymerization and a terpene. In some embodiments, the one or more polycannabinoids and the at least one terpene are formulated for immediate release. In some embodiments, the one or more polycannabinoids and the at least one terpene are formulated for controlled release. Immediate release formulations dissolve without delaying or prolonging dissolution or absorption of the drug. In contrast, controlled release formulations are designed to release the active content in a predictable pattern in vivo over an extended time period, such as over a period greater than an hour.

### Therapeutic Methods

Provided herein are compositions comprising an effective amount of one or more cannabinoid polymer as described herein or salt form thereof, and a pharmaceutically or cosmetically acceptable carrier for the treatment of a condition. Conditions for treatment according to the present disclosure include, without limitation, asthma, autoimmune diseases such as multiple sclerosis, various cancers, ciliopathies, diabetes, heart disease, hypertension, inflammatory bowel disease, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, Turner syndrome.

In some embodiments, provided is a method for treatment of cancer comprising administering a composition comprising a polyCBD described herein. In some embodiments, the cancer is solid cancer or a blood cancer. In some embodiments, the solid cancer is a breast cancer. In some embodiments, the breast cancer is triple negative breast cancer. In some embodiments, the breast cancer is stage 1, 2, 3, or 4 breast cancer. In some embodiments, the breast cancer is advanced stage breast cancer. In some embodiments, the breast cancer is ductal carcinoma in situ (DCIS), invasive breast cancer, triple-negative breast cancer, inflammatory breast cancer, Paget disease of the breast, angiosarcoma, or Phyllodes tumor. In some embodiments, the subject is administered a chemotherapy or radiation therapy prior to, concurrently, or following administration of the composition. In some embodiments, the subject is administered the composition prior to, concurrently, or following administration of the chemotherapy or radiation therapy. Exemplary chemotherapy agents include, anthracyclines, such as doxorubicin (Adriamycin) and epirubicin (Ellence); taxanes, such as paclitaxel (Taxol) and docetaxel (Taxotere); 5-fluorouracil (5-FU) or capecitabine (Xeloda); Cyclophosphamide (Cytoxan); and Carboplatin (Paraplatin). In some embodiments, the chemotherapy agent is gemcitabine or oxaliplatin. In some embodiments, the composition is administered in conjunction with naltrexone. In some embodiments, the composition further comprises naltrexone. In some embodiments, admiration of a composition provided herein provides for a reduction in cancer cell volume and/or mass. In some embodiments, admiration of a composition provided herein provides for increased efficacy of a cancer therapeutic compared to a treatment regimen without administration of the composition. In further embodiments, the cancer therapeutic is an antibody, cell, small peptide, nucleic acid or compound. In some embodiments, the composition comprises a polycannabinoid described herein present in an effective amount for treatment of nausea and vomiting from chemotherapy.

Provided herein a method for treatment of a mental condition. Exemplary mental conditions include, without limitation, depression and anxiety. Provided herein a method for treatment of epilepsy. Provided herein are methods for reduction of anxiety, comprising administering to a subject a composition described herein. Provided herein are methods for reduction of depression, comprising administering to a subject a composition described herein. Reduction of the anxiety and/or depression may be in frequency, duration, or intensity. In some embodiments, a composition described herein is administered to a subject receiving: Ambien (zolpidem), Belsomra (suvorexant), Butisol (butabarbital), Doral (quazepam), Edluar (zolpidem), Estazolam, Flurazepam, Halcion (triazolam), Hetlioz (tasimelteon), Intermezzo (zolpidem), Lunesta (eszopiclone), Restoril (temazepam), Rozerem (ramelteon), Seconal (secobarbital), Silenor (doxepin), Sonata (zaleplon), Zolpimist (zolpidem), Benadryl (diphenhydramine), or Unisom (doxylamine). In some embodiments, a composition described herein is administered to a subject receiving: Piper methysticum L.f. (Piperaceae), Zizyphus jujuba Mill var. spinose (Rhamnaceae), Valeriana officinalis L. (Caprifoliaceae), Hypericum montbretii Spach (Hypericaceae), Pinus massoniana Lamb. (Pinaceae), Scutellaria baicalensis Georgi (Lamiaceae), Atractylodes macrocephala Koidz. (Compositae), Ipomoea orizabensis (Pelletan) Ledeb. ex Steud. (Convolvulaceae), Ternstroemia lineata DC. (Pentaphylacaceae), Rhus parviflora Roxb. (Anacardiaceae), Dimocarpus longan Lour. (Sapindaceae), Crassocephalum bauchiense (Hutch.) Milne-Redh. (Compositae), Chrysanthemum morifolium Ramat. (Compositae), Dorstenia arifolia Lam. (Moraceae), Magnolia officinalis Rehder & E. H. Wilson (Magnoliaceae), Glycyrrhiza glabraL. (Leguminosae), Ecklonia cavaKjellman (Lessoniaceae), or Melissa officinalis L. (Lamiaceae).

Provided herein are methods for reduction of sleep disturbance, comprising administering to a subject a composition described herein. Reduction of the sleep disturbance may be in frequency, duration, or intensity. In some embodiments, a composition described herein is administered to a subject receiving an antidepressant or antihypertensive. In some embodiments, a composition described herein is administered to a subject receiving Fluoxetine, Imipramine, Phenelzine, Protriptyline, Clonidine, Methyldopa, Propranolol, Triamterene, Corticosteroids, Levodopa, Phenytoin, Quinidine, Theophylline, Thyroid hormone. In some embodiments, a composition described herein is administered to a subject receiving caffeine containing products, cough and cold medication, or nasal decongestants. In some embodiments, a composition described herein is administered to a subject receiving an expectorant.

### Additional Uses

In another aspect, the cannabinoid polymers described herein can be used as a biodegradable, compostable, and/or recyclable polymer for the preparation of commodity items. These polymers can be processed using techniques and methods known in the art, e.g., those known for processing polyester polymers.

### Exemplary Embodiments

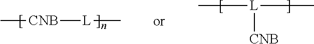

wherein:
CNB is a cannabinoid moiety,
L is a linking group; and
n represents the number of repeat units wherein n is at least 2.

In certain embodiments of the polymer, each cannabinoid moiety is independently derived CBG, CBD, CBC, CBND, DHCBD, CBG-R, CBD-R, CBC-R, CBND-R, or DHCBD-R, wherein the cannabinoid moiety is bound to the linking group via hydroxyl groups, acid groups, or ester groups on the cannabinoid before polymerization.

In other embodiments of the polymer, each cannabinoid moiety has one of the following structures before polymerization, wherein the R group is C1-C10 alkyl optionally substituted with one or more heteroatoms, a heterocycloalkyl group, or a heteroaryl group, specifically C1-C6 alkyl, and more specifically n-pentyl or n-propyl; for the naturally occurring phytocannabinoids like CBD and CBG, R=methyl, ethyl, propyl, butyl, pentyl, hexyl, 4′-(3-carboxypropyl)-, 4′-(4-hydroxybutyl), 1,1-dimethylheptyl, 4′-[2-(1H-1,2,3-triazol-yl)ethyl]-, 4′-(2-morpholinoethyl)-, or 4′-(2-ethoxyethyl)-:

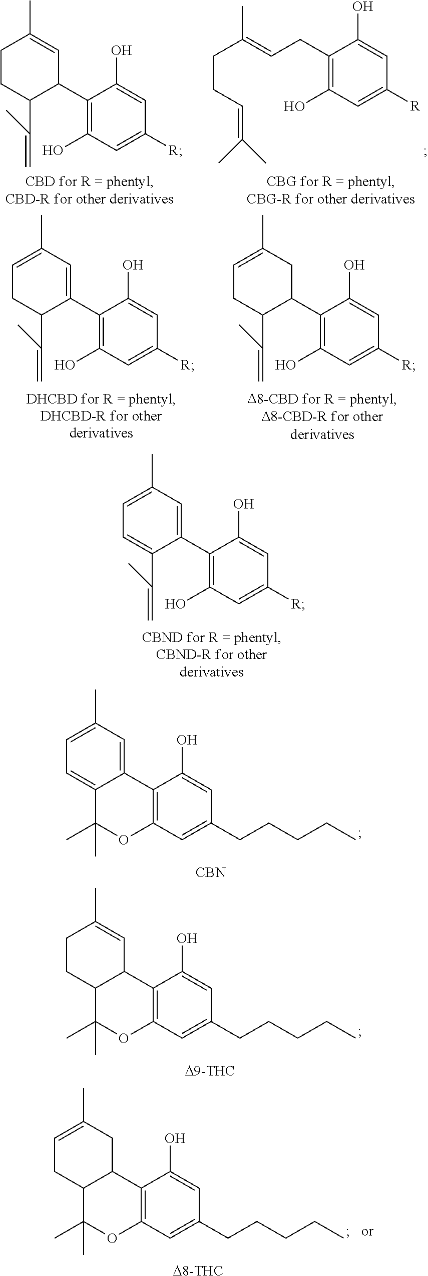

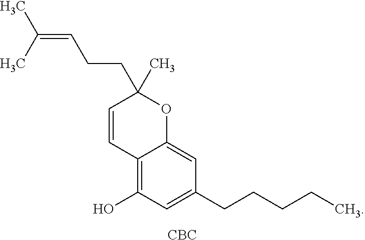

In certain embodiments of the polymer, the linking group is a linear or branched hydrocarbon chains containing from 3 to 50 carbon atoms, optionally interrupted with one or more oxygen atoms or aromatic groups. In certain embodiments, the linking group is interrupted with one or more benzyl groups.

In some embodiments of the polymer, the linking groups are monomers which polymerize to form vinyl polymers, polyurethanes, polyesters, polyethers, polyamides, polyimides, polyamino acids, polypeptides, or polysaccharides, or a combination thereof.

In other embodiments of the polymer, the polymer further comprises an endcapping group, wherein the endcapping group is defined by the type of polymerization and monomers used as is known in the art, or specifically endcapped with a cannabinoid moiety having one hydroxyl group, acid group, or ester group before reaction with the polymer. In certain embodiments, the endcapping group has the following structure before reaction with the polymer:

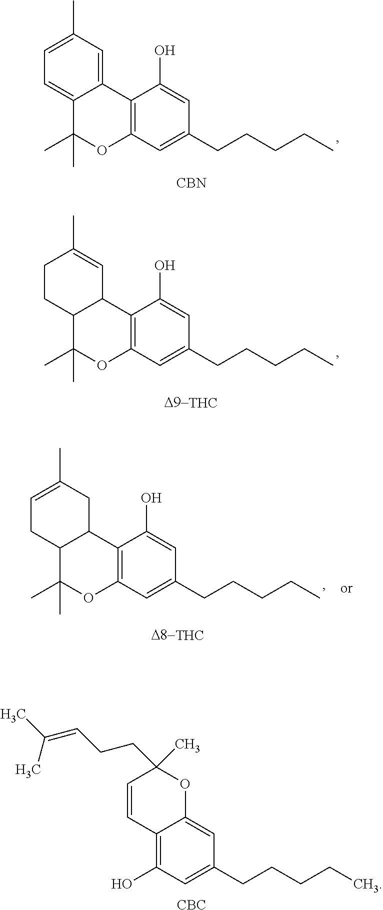

In certain embodiments of the polymer, the number average molecular weight of the polymer is about 1,000 daltons to about 60,000 daltons. In other embodiments of the polymer, the number average molecular weight of the polymer is about 7,000 daltons to about 40,000 daltons.

In another aspect, a pharmaceutical composition comprises a polycannabinoid polymer and a pharmaceutically acceptable carrier. In another aspect, a nutraceutical composition comprises a polycannabinoid polymer and optionally a carrier

In yet another aspect, a method for delivering a cannabinoid to a patient in need thereof, comprises administering to the patient a therapeutically effective amount of a polycannabinoid.

In still another aspect, a method for treating a disease state in a patient, comprises administering to the patient a therapeutically effective amount of a polycannabinoid. In certain embodiments, the disease state is glaucoma, AIDS wasting, neuropathic pain, spasticity associated with multiple sclerosis, fibromyalgia chemotherapy-induced nausea, allergies, inflammation, infection, epilepsy, depression, migraine, bipolar disorders, anxiety disorder, drug dependency, withdrawal syndromes, and chronic pain.

In another aspect, a microsphere comprises the polycannabinoid.

In still another aspect, a film comprises the polycannabinoid.

In yet another aspect, a nonwoven fabric comprises the polycannabinoid.

In another aspect, a wound dressing comprises the polycannabinoid.

In still yet another aspect, a stent a catheter, or a catheter coating comprises the polycannabinoid.

In another aspect, a transdermal patch comprises the polycannabinoid.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

Materials and Methods: Cannabidiol (CBD) was purchased from EcoGen BioSciences and used as received. Cannabigerol (CBG) was purchased from Mile High Labs, Inc. and used as received. All other chemicals were purchased from Sigma Aldrich and used without further purification unless otherwise noted. Unless otherwise indicated, 1H NMR data were collected using a Bruker AVANCE 500 MHz instrument. Thermo Gravimetric Analysis was conducted using a TA Instruments TGA Q-500 and DSC was collected using a TA Instruments DSC Q-20. GPC was taken using a WATERS GPC equipped with a 1515 HPLC Pump and Waters 717Plus Autoinjector. Ultra Performance Liquid Chromatograph tandem Mass Spectrometry (UPLC/MS/MS) was conducted using a Waters Acquity UPLC-TQD equipped with a PDA detector.

Example 1—Preparation of Sebacoyl Chloride: To a flame dried 25 mL round bottom flask was added 5 grams (24.7 mmol) of sebacic acid and 10 mL (137.8 mmol) of thionyl chloride. A reflux condenser was added to the flask and the solution allowed to stir at 90° Celsius for 3 hours until all the solid acid had dissolved. After cooling to room temperature, excess thionyl chloride was removed under vacuum. Five mL of anhydrous toluene was added and removed under vacuum to further remove excess thionyl chloride. The clear yellow solution was further purified by vacuum distillation to yield a colorless oil (5 grams, yield 84.6%).

Example 2A—Preparation of Cannabidiol Polyester-poly(Cannabidiol-Sebacate): To a flame dried 25 mL three-neck round bottom flask, containing a solution of 10 mL anhydrous DCM and 5 mL of anhydrous pyridine, 1 gram (3.2 mmol) of dry CBD was dissolved. Next, 0.68 mL of freshly made and distilled sebacoyl chloride (3.2 mmol) was added dropwise at room temperature over 10 minutes and the reaction allowed to stir at room temperature for 96 hours. The viscous solution was precipitated using dry-ice cold methanol. The solid was collected by filtering and dried under vacuum for 2 days to give 1.3 grams of white polymer; yield 81%. ¹H NMR (400 MHZ, CDCl₃): § 6.68 (s, 2H), 5.20 (s, 1H), 4.54 (s, 1H), 4.46 (s, 1H), 3.51-3.45 (m, 1H), 2.69-2.27 (m, 7H), 2.20-1.97 (m, 2H), 1.89-1.47 (m, 16H), 1.46-1.40 (m, 12H), 0.87 (t, 3H).

Example 2B—Preparation of Cannabidiol Polyester-poly(Cannabidiol-Sebacate): 20 mL of anhydrous methylene chloride (DCM) and 10 mL of anhydrous pyridine were added to a flame-dried 50 mL two-neck round bottom flask. 1.0 gram (0.00318 mol) of cannabidiol (CBD) was added to the solution and allowed to dissolve while stirring. The solution was then chilled to 0° C. in an ice-water bath. 0.68 mL (0.00318 mol) of Sebacoyl Chloride was then added dropwise over 30 minutes and the reaction stirred for 4 days. After some time, the solution turned from cloudy white to a transparent light-yellow. After the reaction finished, it was concentrated and precipitated in cold methanol to give white polymer strands (1.52 g, 86% yield). Mn 28k, PDI 1.52. ¹H NMR (500 MHZ, CDCl₃): δ (ppm): 6.68 (s, 2H), 5.19 (s, 1H), 4.54 (s, 1H), 4.46 (s, 1H), 3.48 (s, 1H), 2.64 (t, 1H), 2.55-2.32 (m, 6H), 2.13 (m, 1H), 2.03-1.99 (m, 1H), 1.81-1.56 (m, 13H), 1.45-1.22 (m, 13H), 0.81 (t, 3H).

Example 3—Preparation of Cannabidiol Polyurethane: To a flame dried three-neck round bottom flask was added 1 gram (3.2 mmol) of dry CBD and 20 mL of anhydrous DCM. Next, 0.456 mL (3.2 mmol) of TDI (tolylene-2,4-diisocyanate) is added to the solution and stirred for 15 minutes. After stirring, 1 mL of a stock solution of DMAP in anhydrous DCM (2 mg/mL) was added to the flask. A reflux condenser was attached to the flask and the solution refluxed for 24 hours. After the reaction finished, the solution was quenched with dry-ice cold methanol. The solid was collected by filtering and dried under vacuum for 2 days to give 1.42 grams of white polymer; yield 84%.

Example 4—Preparation of Cannabidiol Polyester-poly(Cannabidiol-Adipate): Anhydrous methylene chloride (DCM) and anhydrous pyridine were added to a dried 250 mL two-neck round bottom flask. Cannabidiol (CBD) was added to the solution and allowed to dissolve while stirring. The solution was chilled to 0° C. in an ice-water bath. 4 adipoyl chloride was then added dropwise over 30 minutes and the reaction stirred for 4 days. After the reaction was completed, it was concentrated and precipitated in cold methanol to give white polymer strands.

Example 5—Preparation of Cannabigerol Polyester-Preparation of poly(Cannabigerol-Adipate): Anhydrous methylene chloride (DCM) and 40 mL of anhydrous pyridine are added to a dried two-neck round bottom flask. Cannabigerol (CBG) is added to the solution and allowed to dissolve while stirring. The solution is then chilled to 0° C. in an ice-water bath. Adipoyl Chloride is then added dropwise over 30 minutes and the reaction stirred for 4 days. After the reaction is finished, it is concentrated and precipitated in cold methanol to give white polymer strands.

Example 6—Preparation of Cannabigerol Polyester-poly(Cannabigerol-Sebacate): Anhydrous methylene chloride (DCM) and 10 mL of anhydrous pyridine are added to a dried 50 mL two-neck round bottom flask. Cannabigerol (CBG) is added to the solution and allowed to dissolve while stirring. The solution is then chilled to 0° C. in an ice-water bath. 0.67 mL (0.0316 mol) of Sebacoyl Chloride is then added dropwise over 30 minutes and the reaction stirred for 4 days. On day 3, the solution turned from cloudy white to a transparent light-yellow. After the reaction was finished, it was concentrated and precipitated in cold methanol to give white polymer strands.

Example 7—Preparation of Co-Polyester-Preparation of poly(Cannabidiol-co-Cannabigerol-Adipate): Anhydrous chloroform (CDCl₃) and anhydrous pyridine are added to a flame-dried 50 mL two-neck round bottom flask. Cannabigerol (CBG) and Cannabidiol (CBD) are added to the solution and allowed to dissolve while stirring. The solution is then chilled to 0° C. in an ice-water bath. Adipoyl Chloride is then added dropwise over 30 minutes and the reaction stirred for 4 days. After the reaction is finished, it was concentrated and precipitated in cold methanol to give white polymer strands.

Example 8—Preparation of Co-Polyester—Preparation of poly(Cannabidiol-co-Olivetol-Adipate): Anhydrous chloroform (CDCl₃) and Anhydrous pyridine is added to a flame-dried 50 mL two-neck round bottom flask. Cannabidiol (CBD) was added to the solution and allowed to dissolve while stirring. The solution is then chilled to 0° C. in an ice-water bath. 1.165 mL (0.00795 mol) of Adipoyl Chloride is then added dropwise over 30 minutes and the reaction stirred for 4 days. After the reaction is finished, it was concentrated and precipitated in cold methanol to give brown polymer strands.

Example 9—Preparation of Cannabidiol Polyester—poly(Cannabidiol-Terephthalate): 10 mL of anhydrous methylene chloride (DCM) and 10 mL of anhydrous pyridine were added to a flame-dried 50 mL two-neck round bottom flask. 1.0 grams (0.00318 mols) of cannabidiol (CBD) was added to the solution and allowed to dissolve while stirring. The solution was then chilled to 0° C. in an ice-water bath. Terephthaloyl Chloride (0.6456 grams, 0.00318 mols), dissolved in 10 mL of anhydrous DCM, was then added dropwise over 30 minutes and the reaction stirred for 4 days. After the reaction was finished, it was precipitated in cold methanol to give a white, flakey solid (1.45 grams, 88.1% yield).

What is claimed is:

1. A composition, wherein the composition comprises:
a plurality of polymers comprising a first polymer and a second polymer,
wherein the first polymer comprises a plurality of cannabinoid units,
wherein the second polymer comprises up to about 20 cannabinoid units, and
wherein the second polymer comprises less cannabinoid units than the number of units of the first polymer.

2. The composition of claim 1, wherein the second polymer is a bioactive polymer.

3. The composition of claim 1, wherein the second polymer comprises about 2 to 15 cannabinoid units.

4. The composition of claim 1, wherein the second polymer comprises up to about 5, 10, 15, or 18 cannabinoid units.

5. The composition of claim 1, wherein the plurality of polymers comprises a combination of linear and branched polymers.

6. The composition of claim 1, wherein the plurality of polymers are branched polymers.

7. The composition of claim 1, wherein the second polymer has a molecular weight of from about 6 kDa to 8.5 kDa.

8. The composition of claim 1, wherein the first polymer comprises from about 20 to about 80 cannabinoid units.

9. The composition of claim 1, wherein the first polymer has a molecular weight of from about 8 kDa to about 44 kDa.

10. The composition of claim 1, wherein the first polymer and the second polymer are blended together.

11. The composition of claim 1, wherein the second polymer comprises serially linked cannabinoid units.

12. The composition of claim 1, wherein the second polymer further comprises at least one side chain comprising serially linked cannabinoid units.

13. The composition of claim 1, wherein the second polymer comprises an ester linker covalently linking two cannabinoid units.

14. The composition of claim 13, wherein upon degradation of the ester linker a released cannabinoid unit is present in an amount sufficient for a phenolic antioxidant activity.

15. The composition of claim 1, wherein the first polymer and the second polymer comprise from 20 to 50 cannabinoid units combined.

16. The composition of claim 1, wherein the first polymer comprises up to about 50 cannabinoid units.

17. The composition of claim 1, wherein the first polymer comprises between 20 and 40 cannabinoid units.

18. The composition of claim 1, wherein the first polymer comprises between 40 and 50 cannabinoid units.

19. A pharmaceutical composition, wherein the pharmaceutical composition comprises:
the composition of claim 1; and
a pharmaceutically acceptable excipient.

20. A method for delivery of a cannabinoid unit, wherein the method comprises administering to a subject the pharmaceutical composition of claim 19.
